SEQUENCE LISTING (20 sequences)

SEQ ID NO: 1 - DNA, 18 bp, Mus musculus
agtgatgaaagaattact

SEQ ID NO: 2 - DNA, 19 bp, Mus musculus
aataataccacttacaaca

SEQ ID NO: 3 - DNA, 21 bp, Mus musculus
atgaagtgatcaagttcatgg

SEQ ID NO: 4 - DNA, 21 bp, Mus musculus
ggatcttggacaaacaaatgc

SEQ ID NO: 5 - DNA, 20 bp, Mus musculus
tttccatttgatactcttac

SEQ ID NO: 6 - DNA, 20 bp, Mus musculus
tcttagttgctttaccaggg

SEQ ID NO: 7 - DNA, 21 bp, Mus musculus
tgtggttgtaggatataggat

SEQ ID NO: 8 - DNA, 21 bp, Mus musculus
aaaggctttgtgtgaactcgg

SEQ ID NO: 9 - DNA, 25 bp, Mus musculus
agatcttcttcttcaaggaccggtt

SEQ ID NO: 10 - DNA, 22 bp, Mus musculus
ggctggtcagtggcttggggta

SEQ ID NO: 11 - DNA, 26 bp, Mus musculus
gtttttgatgctattgctgagatcca

SEQ ID NO: 12 - DNA, 25 bp, Mus musculus
cccacatttgacgtccagagaagaa

SEQ ID NO: 13 - DNA, 24 bp, Mus musculus
ggcatcctcttgttgctatcactg

SEQ ID NO: 14 - DNA, 24 bp, Mus musculus
gtcatcttgatctcatcccgctgg

SEQ ID NO: 15 - DNA, 24 bp, Mus musculus
ctcgctggacgttggaggaaagaa

SEQ ID NO: 16 - DNA, 24 bp, Mus musculus
agcccatctggtacctgtggttca

SEQ ID NO: 17 - DNA, 20 bp, Mus musculus
cattaacggacaccctgctt

SEQ ID NO: 18 - DNA, 20 bp, Mus musculus
cgtgggacacacatttcaag

SEQ ID NO: 19 - DNA, 20 bp, Mus musculus
agctaggaataatggaatag

SEQ ID NO: 20 - DNA, 21 bp, Mus musculus
aatcaagaacgaaagtcggag

(19) United States
(12) Patent Application Publication
(10) Pub. No.: US 2014/0011822 A1
(43) Pub. Date: Jan. 9, 2014

(54) METHODS AND MATERIALS FOR REDUCING VENOUS STENOSIS FORMATION OF AN ARTERIOVENOUS FISTULA OR GRAFT

(71) Applicant: Sanjay Misra, Rochester, MN (US)

(72) Inventor: Sanjay Misra, Rochester, MN (US)

(21) Appl. No.: 14/005,002

(22) PCT Filed: Mar. 16, 2012
(86) PCT No.: PCT/US2012/029514

§ 371 Date: Sep. 13, 2013

Related U.S. Application Data
(60) Provisional application No. 61/453,457, filed on Mar. 16, 2011.

(51) Int. Cl. A61K 31/366; A61M 37/00

(52) U.S. Cl. CPC A61K 31/366; A61M 37/00
USPC 514/266.24; 514/460; 514/339; 514/414; 514/346; 514/338; 514/275; 514/369; 604/500

(57) ABSTRACT

This document provides methods and materials for reducing venous stenosis formation of an arteriovenous fistula or graft. For example, methods and materials for using VEGF inhibitors to reduce venous stenosis formation of arteriovenous fistulas or grafts are provided.

### CROSS-REFERENCE TO RELATED APPLICATIONS

This application claims the benefit of U.S. Provisional Application Ser. No. 61/453,457, filed Mar. 16, 2011. The disclosure of the prior application is considered part of (and is incorporated by reference in) the disclosure of this application.

### STATEMENT AS TO FEDERALLY SPONSORED RESEARCH

This invention was made with government support under grant number HL098967, awarded by the National Institutes of Health. The government has certain rights in the invention.

### BACKGROUND

1. Technical Field

This document relates to methods and materials involved in reducing venous stenosis formation of an arteriovenous fistula or graft. For example, this document provides methods and materials for using VEGF inhibitors to reduce venous stenosis formation of arteriovenous fistulas or grafts.

2. Background Information

In the United States, more than 400,000 patients have end-stage renal disease (ESRD) and require chronic hemodialysis. Moreover, it is estimated that the population of patients requiring dialysis for renal replacement therapy will double in the coming decade (Collins et al., Am. J. Kidney Dis., 42:A5-7 (2003)). Vascular access through an arteriovenous fistula (AVF) or graft is required for the optimal hemodialysis and clearance of uremic toxins. Unfortunately, AVF failure occurs frequently due to venous stenosis formation. The patency of AVFs at one year is estimated to be only 62% (Rooijens et al., European Journal of Vascular and Endovascular Surgery, 28:583-589 (2004)). Over a billion dollars are spent annually to maintain the function of hemodialysis AVFs and grafts (Collins et al., Am. J. Kidney Dis., 42:A5-7 (2003)). The first line of treatment at the time of stenotic vascular AVF or graft is angioplasty, but recent studies have demonstrated that at six months venous stenosis recurs in a significant fraction of AVFs treated by angioplasty (Misra et al., Kidney Int., 70(11):2006-13 (2006) and Haskal et al., N. Engl. J. Med., 362:494-503 (2010)).

Atrerial and venous smooth muscle cells have many differences as demonstrated in the following references: Deng et al., Arterioscler. Thromb. Vasc. Biol., 26(5):1058-65 (2006); Aitsebaomo et al., Circ. Res., 103(9):929-39 (2008); Wong et al., Cardiovasc. Res., 65(3):702-10 (2005); Kim et al., J. Lab. Clin. Med., 144(3):156-62 (2004); Li et al., J. Cell. Biochem., 99(6):1553-63 (2006); and Turner et al., J. Vasc. Surg., 45(5):1022-8 (2007)).

### SUMMARY

This document provides methods and materials for reducing venous stenosis formation of an arteriovenous fistula or graft. For example, this document provides methods and materials for using VEGF inhibitors to reduce venous stenosis formation of arteriovenous fistulas or grafts. As described herein, delivering a VEGF inhibitor to the adventitia of a vein of an arteriovenous fistula or graft from a position outside the vein can reduce venous stenosis formation. Having the ability to reduce venous stenosis formation of an arteriovenous fistula or graft using the methods and materials provided herein can allow clinicians and patents to maintain the function of arteriovenous fistulas or grafts whether involved in hemodialysis or other types of grafting procedures.

The methods and materials provided herein can be used to reduce venous stenosis formation after surgery, after a biopsy, or after a radiation treatment or exposure. In some cases, the methods and materials provided herein can used to reduce gastrointestinal stensosis after surgery, after a biopsy, or after a radiation treatment or exposure. In some cases, the methods and materials provided herein can used in conjunction with angioplasty or stent placement. For example, the materials provided herein can be delivered using an endovascular catheter configured to target the adventitia. In some cases, the methods and materials provided herein can with an endovascular delivery to the endothelium with or without using angioplasty, stents, or nanaoparticles.

In some cases, a compound (e.g., simvastatin or another statin compound) having the ability to reduce VEGF-A expression can be used as described herein alone or in combination with a VEGF inhibitor to reduce venous stenosis formation of arteriovenous fistulas or grafts. For example, systemic delivery of simvastatin (or another statin such as atorvastatin (Lipitor and Torvast), fluvastatin (Lescol), lovastatin (Mevacor, Altocor, Altoprev), pitavastatin (Livalo, Pitava), pravastatin (Pravachol, Selektine, Lipostat), or rosuvastatin (Crestor)) can be used to reduce venous stenosis formation of arteriovenous fistulas or grafts. In some cases, simvastatin can be used to reduce VEGF-A and MMP-9 gene expression via systemic delivery of simvastatin prior to the placement of an AVF, thereby leading to reduced profibrosis. A reduction in profibrosis can ameliorate venous neointimal hyperplasia and can be accompanied by positive vascular remodeling.

In general, one aspect of this document features a method for reducing venous stenosis formation of an arteriovenous fistula or graft in a mammal. The method comprises, or consists essentially of, administering a VEGF inhibitor to an adventitia of a vein of the arteriovenous fistula or graft from a position outside the vein under conditions wherein venous stenosis formation of the arteriovenous fistula or graft is reduced. The mammal can be a human. The inhibitor can be thalidomide, lapatinib, sunitinib, sorafenib, axitinib, pazopanib, or thiazolidinediones. The VEGF inhibitor can be administered using a sustained release device positioned outside of the vein. The sustained release device can comprise chitosan, alginates, polyethylene glycol, poly lactic acid, copoly lactic acid/glycolic acid, dextrans, acrylates, cyclodextrins, caprolactones, block copolymers, or combinations thereof. The VEGF inhibitor can be administered using a cuff device configured to at least partially surround the vein, wherein the cuff comprises an outlet configured to allow the VEGF inhibitor to be exit the cuff device and contact the adventitia of the vein. The cuff device can be attached to a pump configured to pump the VEGF inhibitor from a reservoir to the outlet. The cuff device can comprise polyethylene, polypropylene, polyimides, polyamides, polystyrene, polytetrafluoroethylene (ePTFE), or a combination thereof. The VEGF inhibitor can be administered using an implantable pump device configured to pump the VEGF inhibitor from a reservoir to an outlet such that the VEGF inhibitor contacts the adventitia of the vein.

In another aspect, this document features a method for reducing venous stenosis formation of an arteriovenous fistula or graft in a mammal. The method comprises, or consists essentially of, administering a statin to an adventitia of a vein of the arteriovenous fistula or graft from a position outside the vein under conditions wherein venous stenosis formation of the arteriovenous fistula or graft is reduced. The mammal can be a human. The statin can be simvastatin. The statin can be administered using a sustained release device positioned outside of the vein. The sustained release device can comprise chitosan, alginates, polyethylene glycol, poly lactic acid, copoly lactic acid/glycolic acid, dextrans, acrylates, cyclodextrins, caprolactones, block copolymers, or combinations thereof. The statin can be administered using a cuff device configured to at least partially surround the vein, wherein the cuff comprises an outlet configured to allow the statin to be exit the cuff device and contact the adventitia of the vein. The cuff device can be attached to a pump configured to pump the statin from a reservoir to the outlet. The cuff device can comprise polyethylene, polypropylene, polyimides, polyamides, polystyrene, polytetrafluoroethylene (ePTFE), or a combination thereof. The statin can be administered using an implantable pump device configured to pump the statin from a reservoir to an outlet such that the statin contacts the adventitia of the vein.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-F relate to the study design. FIG. 1A is a schematic representation of the different procedures used during the study. FIG. 1B depicts a nephrectomy procedure. FIG. 1C depicts fistula or graft placement. FIG. 1D is a schematic of the trial design. FIGS. 1E and 1F are graphs plotting the mean BUN and creatinine levels after nephrectomy. Each bar shows mean±SEM of 4-6 animals per group. A significant difference from control value was indicated by *P<0.05, **P<0.01, or #P<0.001.

FIG. 2. LV-shRNA-VEGF-A transfection decreases VEGF-A expression in cells. Gene expression of VEGF-A (A) and Western blot for VEGF-A (B) in normal AKR-2B, C (control, AKR-2B transfected with control shRNA), or LV (LV-shRNA-VEGF-A transfected cells) demonstrates a greater than two fold decrease in VEGF-A expression with LV-shRNA-VEGF-A silencing when compared to controls.

FIG. 3. VEGF-A expression is reduced in LV-shRNA-VEGF-A transfected vessels. (A) first column shows confocal microscopy for localization of eGFP tagged to LV-shRNA or control shRNA at three days after delivery to the outflow vein (D3) and seven days after delivery (D7). Panel A, second column shows confocal microscopy for localization of α-SMA plus HIF-1α (pink) in LV-shRNA or control shRNA at day 3 and 7. There is a decreased expression of cells staining positive for α-SMA plus HIF-1α in the LV-shRNA-VEGF-A transfected vessels when compared to controls. Panel A, third column shows confocal microscopy for localization of eGFP, α-SMA, and HIF-1α (white) in LV-shRNA-VEGF-A or control shRNA at day 3 and 7. There is a decreased expression of cells staining positive for eGFP, α-SMA, and HIF-1α in the LV-shRNA-VEGF-A transfected vessels when compared to controls. Panel A, fourth to sixth columns show in situ hybridization for mRNA for VEGF-A in the LV-shRNA-VEGF-A-transfected vessels when compared to controls with arrows on cells positive for VEGF-A mRNA expression (brown). By day 3, there was a reduction of mRNA for VEGF-A being localized to the media and adventitia, and by day 7, it was localized to the media and intima (B). In contrast, the vessels transfected with control shRNA exhibited increased mRNA expression of VEGF-A in the adventitia and media by day 3, and in the media and intima by day 7 (B). Panel C reveals the mean gene expression of VEGF-A at day 7 (D7), day 14 (D14), and day 28 (D28) showing significant reduction in VEGF-A expression in the LV-shRNA-VEGF-A transfected vessels when compared to control vessels at days 7 and 14. By day 28, there was a recovery of the expression of VEGF-A in the LV-transfected vessels when compared to controls (P<0.05). Each bar shows mean±SEM of 4-6 animals per group (C). Significant difference from control value was indicated by *P<0.05, **P<0.01, or #P<0.001.

FIG. 4. Confocal microcopy of eGFP, α-SMA, and HIF-1α in LV-shRNA-VEGF-A transfected and control vessels. Panel A, first column shows confocal microscopy for localization of eGFP tagged to LV-shRNA (LV) or control shRNA (C) at three days after delivery to the outflow vein (D3) and seven days after delivery (D7). Panel A, second column shows confocal microscopy for localization of α-SMA in LV-shRNA or control shRNA transfected vessels at day 3 and 7. Panel A, third column shows confocal microscopy for localization of HIF-1α in LV-shRNA-VEGF-A or control shRNA transfected vessels at day 3 and 7. Panel A, fourth column shows confocal microscopy for localization of HIF-1α and α-SMA in LV-shRNA-VEGF-A transfected vessels or control shRNA at day 3 and 7. Panel A, fifth column shows confocal microscopy for localization of eGFP, HIF-1α and α-SMA in LV-shRNA-VEGF-A transfected vessels or control shRNA at day 3 and 7. Panel B depicts a semiquantitative analysis of cells staining positive for HIF-1α and α-SMA and demonstrated a significant reduction in the LV-shRNA-VEGF-A transfected vessels when compared to controls at day 3 and 7. Panel C depicts a semiquantitative analysis of cells staining positive for eGFP, HIF-1α and α-SMA and demonstrated a significant reduction in the LV-shRNA-VEGF-A transfected vessels when compared to controls at day 3 and 7. Significant difference from control value was indicated by *P<0.05, **P<0.01, or #P<0.001.

FIG. 5. Hematoxylin and eosin (H and E) staining of the LV-shRNA-VEGF-A transfected vessels showing reduced VNH formation. Representative sections after hematoxylin and eosin (H and E) staining at the venous stenosis of the LV-shRNA-VEGF-A (LV) and scrambled-VEGF-A (C) transfected animals at day 3 (D3), day 7 (D7) (Panel A), day 14 (D14), and day 28 (D28) (Panel B) after the creation of the arteriovenous fistula or graft.

FIG. 6. Semiquantitative analysis shows significant reduction in VNH formation in the LV-shRNA-VEGF-A transfected vessels. Semiquantitative analysis for wall area (Panel A) reveals a significant decrease in the wall area of LV-shRNA-VEGF-A (LV) when compared to scrambled-VEGF-A (C) for days 3-14. Semiquantitative analysis for lumen vessel area (Panel B) reveals a significant increase in the mean lumen vessel area of LV transfected vessels when compared to C for days 3-14. Semiquantitative analysis for cellular density (Panel C) reveals a significant decrease in the mean cell density of LV transfected vessels when compared to C for days 3-14. A typical ultrasound of the outflow vein is shown in Panel D with diameter measurements for the outflow vein in Panel E. Semiquantitative analysis for pooled outflow vein diameter measurements (Panel F) reveals a significant increase in the mean outflow vein diameter of LV transfected vessels when compared to C by day 14. A significant difference from control value was indicated by *P<0.05, **P<0.01, or #P<0.001. Each bar shows mean±SEM of 4-6 animals per group (Panels A-C).

FIG. 7. Apoptosis is increased in the LV-sh-RNA-VEGF-A transfected vessels. Panel A depicts TUNEL staining at the venous stenosis of the LV-shRNA-VEGF-A (LV) and scrambled-VEGF-A (C) at 14 (D14), and 28 (D28) after the creation of the arteriovenous fistula or graft. Cells staining brown are positive for α-SMA. All are 40×. Pooled data for lentivirus and scrambled groups each at day 14 and 28 is shown in Panel B. This demonstrates a significant increase in the mean TUNEL staining at day 14. Pooled data for the lentivirus and scrambled groups for caspase 3 activity at days 3, 7, and 14 is shown in Panel C. This demonstrates a significant increase in the mean caspase 3 activity at day 14. A significant difference from control value was indicated by *P<0.05, **P<0.01, or #P<0.001. Each bar shows mean±SEM of 3-4 animals per group (Panels A-C).

FIG. 8. Cellular Proliferation is decreased in LV-shRNA-VEGF-A transfected vessels. Panel A depicts Ki-67 staining at the venous stenosis of the LV-shRNA-VEGF-A (LV) and control shRNA (C) 14 (D14), and 28 (D28) days after the creation of the arteriovenous fistula or graft. Cells staining brown are positive for α-SMA. All are 40×. Pooled data for the LV-shRNA-VEGF-A and control shRNA groups each at day 14 and 28 is shown in Panel B. This demonstrates a significant decrease in the mean amount of Ki-67 staining in the LV-shRNA-VEGF-A transfected vessels when compared to control shRNA at day 7 and 14. A significant difference from control value was indicated by *P<0.05, **P<0.01, or #P<0.001. Each bar shows mean±SEM of 4 animals per group (Panels A-B).

FIG. 9. Gene expression of MMP-2, MMP-9, TIMP-1, TIMP-2, and ADAMTS-1 is reduced in LV-shRNA-VEGF-A transfected vessels. RT-PCR analysis for MMP-2 (Panel A), MMP-9 (Panel B), TIMP-1 (Panel E), TIMP-2 (Panel F), and ADAMTS-1 (Panel G) expression after transfection with either LV-shRNA-VEGF-A (LV) or control shRNA (C) at 7 (D7), 14 (D14), and 28 days (D28) after AVF placement. A typical zymogram for MMP-2 (Panel C) and MMP-9 (Panel D) is shown in the upper panel and the pooled data on the lower panels. This demonstrates a significant reduction in the average amount of MMP-2, MMP-9, TIMP-1, and TIMP-2 expression in the LV-shRNA-VEGF-A transfected vessels when compared to control shRNA at day 7. ADAMTS-1 expression was decreased significantly at day 7 and 14 in the LV-shRNA-VEGF-A transfected vessels when compared to controls. By day 28, there is a significant increase in MMP-2 expression in the LV-shRNA-VEGF-A transfected vessels when compared to controls (P<0.05). By zymography, pro-MMP-9 expression was significantly decreased by day 14 while at day 7, MMP-2 and MMP-9 was decreased. Data are mean±SEM. A significant difference from control value was indicated by *P<0.05, **P<0.01, or #P<0.001. Each bar shows mean±SEM of 3-6 animals per group (Panels A-G).

FIG. 10. Smooth muscle cell density is reduced in LV-shRNA-VEGF-A transfected vessels. Panel A depicts α-SMA staining at the venous stenosis of the LV-shRNA-VEGF-A (LV) and control shRNA (C) at 14 (D14), and 28 days (D28) after the placement of the AVF. Cells staining pink are positive for α-SMA. All are 40×. Pooled data for the LV-shRNA-VEGF-A and control shRNA groups at day 14 and 28 are shown in Panel B. This demonstrates a significant reduction in average staining for α-SMA in LV-shRNA-VEGF-A transfected vessels when compared to control shRNA by day 14. Panel C contains results from an RT-PCR analysis for VEGFR-1 expression after transfection LV-shRNA-VEGF-A (LV) and control shRNA day 7 (D7), 14 days (D14), and 28 days (D28) after AVF placement. A typical blot is shown in the upper panel, and the pooled data is shown on the lower panel. This demonstrates a significant reduction in VEGFR-1 expression in the LV-shRNA-VEGF-A transfected vessels when compared to control shRNA at day 7. A significant difference from control value was indicated by *P<0.05, **P<0.01, or #P<0.001. Each bar shows mean±SEM of 4-6 animals per group (Panels A-C).

FIG. 11. There is decreased hypoxyprobe staining and HIF-1α expression in LV-shRNA-VEGF-A transfected vessels. Panel A depicts hypoxyprobe staining at the venous stenosis of LV-shRNA-VEGF-A (LV) and control shRNA (C) at 14 (D14), and 28 (D28) after the placement of the arteriovenous fistula or graft. Brown staining cells are positive for hypoxyprobe. All are 40×. Pooled data for the LV-shRNA-VEGF-A and control groups at day 14 and 28 is shown in Panel B. This demonstrates a significant reduction in the average hypoxyprobe staining in the LV-shRNA-VEGF-A transfected vessels when compared to controls at day 14. Panel C contains results from an RT-PCR analysis for HIF-1α expression after transfection LV-shRNA-VEGF-A and control shRNA at day 7 (D7), 14 (D14), and 28 (D28) after placement of AVF. A typical blot is shown in the upper panel, and the pooled data is shown in the lower panel. This demonstrates a significant reduction in HIF-1α expression in the LV-shRNA-VEGF-A transfected vessels when compared to controls at days 7 and 14. A significant difference from control value was indicated by *P<0.05, **P<0.01, or #P<0.001. Each bar shows mean±SEM of 4-6 animals per group (Panels A-C).

FIG. 12. There is decreased proliferation, invasion, α-SMA, and MMP-2 expression with increased caspase 3 activity in the LV-shRNA-VEGF-A transfected cells subjected to hypoxia. Western blot was performed for α-SMA after transfection LV-shRNA-VEGF-A (LV) and scrambled shRNA-VEGF-A (C) in AKR-2B Fibroblasts subjected to hypoxia at 24 (24 h) and 72 hours (72 h). A typical Western blot is shown in the upper panel of Panel A, and the pooled data on the lower panel of Panel A. This demonstrates a significant reduction in α-SMA expression in the LV-shRNA-VEGF-A transfected cells when compared to controls at 24 and 72 hours. Panel B shows staining for α-SMA (red positive cells) at 24 and 72 hours. Panel C contains the pooled data for the average intensity of cells staining positive for α-SMA demonstrating a significant decrease in the LV-shRNA-VEGF-A transfected cells when compared to control cells at both 24 and 72 hours. Panel D contains results from an invasion assay for LV-shRNA-VEGF-A and control cells for normoxia and hypoxia. Panel E contains the pooled data for invasion for the LV-shRNA-VEGF-A and control cells showing a significant decrease in both the normoxic and hypoxic groups. Panel F contains the pooled data for proliferation for the LV-shRNA-VEGF-A and control transfected cells showing a significant decrease in both the normoxic and hypoxic groups. Panel G is a zymogram of cells after transfection LV-shRNA-VEGF-A and control shRNA in AKR-2B fibroblasts subjected to hypoxia at 24 and 72 hours. This demonstrates a significant reduction in both pro and active MMP-2 activity at 24 hours for the LV-shRNA-VEGF-A when compared to control cells. Panel H contains pooled data of caspase 3 activity after transfection of LV-shRNA-VEGF-A or control shRNA in AKR-2B fibroblasts subjected to hypoxia at 24 and 72 hours showing a significant increase in the mean caspase 3 activity in the LV-shRNA-VEGF-A transfected cells when compared to controls at 24 and 72 hours.

FIG. 13 is a schematic of a proposed mechanism. The schematic shows normal vein (A), vein after AFV placement (B), and outflow vein after fistula or graft placement with LV-shRNA-VEGF-A silencing and its different mechanisms (C).

FIG. 14 contains representative ultrasound images from the outflow vein of animals sacrificed at day 3 (D3), 7 (D7), 14 (D14), and 28 (D28).

FIG. 15 is a side view of an arteriovenous fistula or graft with a device configured to release a VEGF inhibitor.

FIG. 16 is a side view of an arteriovenous fistula or graft with a device having a cuff configured to release a VEGF inhibitor.

FIG. 17 is a side view of an arteriovenous fistula or graft with a pump device configured to deliver a VEGF inhibitor.

FIG. 18 (A) shows the nephrectomy procedure while (B) demonstrates the placement of the AVF. (C) is the study schema. (D) is the average BUN values after nephrectomy. SV is simvastatin group, and C is the control group. There was a significant increase in the average BUN values after nephrectomy for the control and simvastatin group (P<0.001). For the simvastatin group when compared to controls, at 8 weeks, there was a significant decrease in the mean BUN (P<0.01). (E) is the average creatinine values after nephrectomy. There was a significant decrease in the average creatinine values after nephrectomy for the control and simvastatin groups at 8-weeks (P<0.01). For the simvastatin group when compared to controls, at 8 weeks, there was a significant decrease in the mean creatinine values (P<0.001). Each bar represents mean±SEM of 4-6 animals. Significant differences between simvastatin treated and controls is indicated by *P<0.01 or **P<0.001.

FIG. 19. Gene expression of VEGF-A and MMP-9 are reduced in simvastatin treated vessels when compared to controls. RT-PCR analysis of VEGF-A (A) and MMP-9 (B) expression after treatment with either control (C) or simvastatin (SV) at day 7 or 14 after AVF placement. A typical blot is shown in the upper panel and the pooled data in the lower panel. (A) shows that the average VEGF-A expression is significantly decreased in the simvastatin treated vessels when compared to controls by day 7 and 14 (both P<0.01). (B) shows that the average MMP-9 expression is significantly decreased at day 7 (P<0.0001) and 14 (P<0.0001) in the simvastatin treated vessels when compared to controls. Each bar represents mean±SEM of 3-5 animals. Significant differences between simvastatin treated and controls is indicated by *P<0.01 and ^{#}P<0.0001.

FIG. 20. Gene expression of VEGF-A, MMP-2, and MMP-9 is reduced in simvastatin treated kidneys when compared to controls. RT-PCR analysis of VEGF-A (A), MMP-2 (B), and MMP-9 (C) expression after treatment with either control (C) or simvastatin (SV) at day 7 or 28 after AVF placement. A typical blot is shown in the upper panel, and the pooled data is shown in the lower panel. (A) shows that the average VEGF-A expression is significantly decreased at day 28 in the simvastatin treated kidneys when compared to controls (P<0.01) while in (B), there is a significant reduction in the average MMP-2 expression in the simvastatin treated kidneys when compared to controls (P<0.001). (C) shows that the average MMP-9 is significantly decreased at day 7 (P<0.001) and 28 (P<0.0001) in the simvastatin treated kidneys when compared to controls. Each bar represents mean±SEM of 3-5 animals. Significant differences between simvastatin treated and controls is indicated by *P<0.01, **P<0.001, and ^{#}P<0.0001.

FIG. 21. Hematoxylin and eosin (H and E) staining of the simvastatin treated vessels showing reduced venous neointimal hyperplasia and positive vascular remodeling. (A) is the representative sections after hematoxylin and eosin (H and E) staining at the venous stenosis of either control (C) or simvastatin (SV) at day 14 or 28 after AVF placement, and in animals without nephrectomy at day 28 (normal) after the placement of the AVF. The upper panels are 10×, and the lower panels are 40×. L is the lumen. NI is the neointima, and ADV is the adventitia/media. (B) is the semiquantitative analysis, which shows a significant decrease in the average area of the neointima of the simvastatin treated vessels (SV) when compared to control (C) group for day 14 (P<0.0001) and 28 (P<0.001). (C) is the semiquantitative analysis, which shows a significant decrease in the average area of the media/adventitia of the simvastatin treated vessels (SV) when compared to control (C) group for day 14. (D) demonstrates a significant increase in the average lumen vessel area of the simvastatin treated vessels (SV) when compared to control (C) group for day 14 and 28 (P<0.001). (E) shows a significant decrease in the average cell density in the neointima of the simvastatin treated vessels (SV) when compared to control (C) group for day 14 (P<0.0001), 28 (P<0.001), and day 28 normal (P<0.01). (F) shows similar results in that there is a significant reduction in the average cell density in the media/adventitia of the simvastatin treated vessels (SV) when compared to control (C) group for day 14 (P<0.001), 28 (P<0.01), and day 28 normal (P<0.001). Each bar represents mean±SEM of 3-4 animals. Significant differences between simvastatin treated and controls is indicated by *P<0.01, **P<0.001, or ^{#}P<0.0001.

FIG. 22. TUNEL staining is increased in simvastatin treated vessels. (A) is the representative sections after TUNEL staining at the venous stenosis after treatment with either control (C) or simvastatin (SV) at day 14 or 28 after AVF placement, and in animals without nephrectomy at day 28 (normal) after the placement of the AVF. Cells staining brown are positive for TUNEL. Upper panel is 40× and the lower panel is a magnification view of the box. (B) is the semiquantitative analysis, which shows a significant increase in the average TUNEL staining in the simvastatin treated vessels (SV) when compared to control (C) group for day 14 (P<0.0001) and 28 (P<0.0001). Each bar represents mean±SEM of 3 animals. Significant differences between simvastatin treated and controls is indicated by ^{#}P<0.0001.

FIG. 23. Cellular proliferation is decreased in simvastatin treated vessels. (A) is the representative sections after Ki-67 staining at the venous stenosis after treatment with either control (C) or simvastatin (SV) at day 14 or 28 after AVF placement, and in animals without nephrectomy at day 28 (normal) after the placement of the AVF. Upper panel is 40×, and the lower panel is a magnification view of the box. Brown staining nuclei are positive for Ki-67. IgG negative controls are shown. (B) is the semiquantitative analysis, which shows a significant decrease in the average Ki-67 staining in the simvastatin treated vessels (SV) when compared to control (C) group for day 14 (P<0.001), 28 (P<0.0001), and day 28 normal (P<0.001). Each bar represents mean±SEM of 3-4 animals. Significant differences between simvastatin treated and controls is indicated by **P<0.001, and ^{#}P<0.0001.

FIG. 24. Smooth muscle cell density is decreased in simvastatin treated vessels. (A) is the representative sections after α-smooth muscle cell actin staining at the venous stenosis after treatment with either control (C) or simvastatin (SV) at day 14 or 28 after AVF placement, and in animals without nephrectomy at day 28 (normal) after the placement of the AVF. Upper panel is 40×, and the lower panel is a magnification view of the box. Brown staining cytoplasm is positive for α-smooth muscle cell actin. (B) is the semiquantitative analysis which shows a significant decrease in the average α-smooth muscle cell actin staining in the simvastatin treated vessels (SV) when compared to control (C) group for day 14 (P<0.0001), 28 (P<0.0001), and day 28 normal (P<0.01). Each bar represents mean±SEM of 3-4 animals. Significant differences between simvastatin treated and controls is indicated by *P<0.01 and ^{#}P<0.0001.

FIG. 25. Gene expression of CTGF and Picrosirius red staining are reduced in simvastatin treated vessels when compared to controls. (A) is RT-PCR analysis of CTGF expression after treatment with either control (C) or simvastatin (SV) at day 7 or 14 after AVF placement. A typical blot is shown in the upper panel, and the pooled data are shown in the lower panel. (A) shows that the average CTGF expression is significantly decreased at 14 in the simvastatin treated vessels when compared to controls (P<0.001). (B) shows representative sections after Picrosirius red staining at the venous stenosis after treatment with either control (C) or simvastatin (SV) at day 14 or 28 after AVF placement, and in animals without nephrectomy at day 28 (normal) after the placement of the AVF. Upper panel is 40×, and the lower panel is a magnification view of the box. More intense red staining is representative of collagen 1 and 3 staining. Qualitatively, there are decreased Sirius red staining in the simvastatin treated vessels when compared to controls. Each bar represents mean±SEM of 3-5 animals. Significant differences between simvastatin treated and controls is indicated by *P<0.01.

FIG. 26. HIF-1α expression and hypoxyprobe staining are reduced in simvastatin treated vessels when compared to controls. (A) is RT-PCR analysis of HIF-1α expression after treatment with either simvastatin (SV) or control (C) at day 7 or 14 after AVF placement. A typical blot is shown in the upper panel and the pooled data in the lower panel. (A) shows that the average HIF-1α expression is significantly decreased at day 7 (P<0.001) and 14 (P<0.0001) in the simvastatin treated vessels when compared to controls. (B) is hypoxyprobe staining at the venous stenosis after treatment with either control (C) or simvastatin (SV) at day 14 or 28 after AVF placement. Cells staining brown are positive for hypoxyprobe. IgG negative controls are shown. (C) shows that the average staining for hypoxyprobe is significantly in the simvastatin treated vessels when compared to controls at day 14 (P<0.001) and 28 (P<0.01). Each bar represents mean±SEM of 3-5 animals. Significant differences between simvastatin treated and controls is indicated by *P<0.01 or **P<0.001.

FIG. 27. There is decreased α-smooth muscle cell expression, migration, and proliferation with increased caspase 3 activity in simvastatin treated cells subjected to hypoxia. (A) Left panel shows a typical Western blot for α-smooth muscle cell actin after treatment with either control (C) or simvastatin (SV) in NIH 3T3 cells subjected to normoxia or hypoxia for 24 hours. Right panel is the pooled data for three separate experiments for three concentrations of simvastatin (1 μM, 5 μM, and 10 μM). This demonstrates a significant reduction in the average α-smooth muscle cell actin expression for 10 μM concentration of simvastatin at 24 hours of hypoxia (P<0.01). (B) is confocal microscopy for α-smooth muscle cell actin staining and phalloidin in NIH 3T3 cells at 24 hours of hypoxia or normoxia. Red staining cells are positive for α-smooth muscle cell actin, phalloidin is green, and the nuclei are blue. This demonstrates a significant reduction in co-staining for α-smooth muscle cell actin and phalloidin in the 5 and 10 μM concentrations of simvastatin at 24 hours of hypoxia (P<0.0001, both concentrations) and normoxia (P<0.01 for 5 μM and P<0.0001 for 10 μM). (C) left panel is a representative picture from a migration experiment. The right panel is the pooled migration data for three separate experiments for three concentrations of simvastatin (1 μM, 5 μM, and 10 μM). There is a significant reduction in the migration of NIH 3T3 cells for the 5 μM and 10 μM concentrations of simvastatin subjected to 24 hours hypoxia (P<0.0001, both concentrations) and normoxia (P<0.01 for the 5 μM and P<0.0001 for 10 μM). (D) is the pooled proliferation data for three separate experiments for three concentrations of simvastatin (1 μM, 5 μM, and 10 μM). This demonstrates that there is a significant reduction in the proliferation of NIH 3T3 cells subjected to 24 hours hypoxia and normoxia for the 5 μM and 10 μM concentrations of simvastatin (P<0.0001). (E) is the pooled caspase 3 activity data for three separate experiments for three concentrations of simvastatin (1 μM, 5 μM, and 10 μM). This demonstrates that there is a significant increase in the caspase 3 activity of NIH 3T3 cells subjected to 24 hours hypoxia for 5 μM and 10 μM concentrations of simvastatin (P<0.0001). Each bar represents mean±SEM of three experiments. Significant differences between simvastatin treated and controls is indicated by *P<0.01, **P<0.001, or ^{#}P<0.0001.

### DETAILED DESCRIPTION

This document provides methods and materials for reducing venous stenosis formation of an arteriovenous fistula or graft. For example, this document provides methods and materials for using VEGF inhibitors to reduce venous stenosis formation of arteriovenous fistulas or grafts. As described herein, delivering a VEGF inhibitor to the adventitia of a vein of an arteriovenous fistula or graft from a position outside the vein can reduce venous stenosis formation.

Any type of mammal having an arteriovenous fistula or graft can be treated as described herein. For example, humans, monkeys, dogs, cats, horses, cows, pigs, sheep, rats, and mice having an arteriovenous fistula or graft can be treated with one or more VEGF inhibitors. Examples of VEGF inhibitors include, without limitation, anti-VEGF antibodies, RNAi molecules designed to inhibit VEGF expression, thalidomide, lapatinib (Tykerb), sunitinib (Sutent), sorafenib (Nexavar), axitinib, pazopanib, thiazolidinediones, and avastin. In some cases, one or more than one VEGF inhibitor (e.g., two, three, four, five, or more VEGF inhibitors) can be administered to a mammal having an arteriovenous fistula or graft to reduce venous stenosis formation.

Any appropriate method can be used to identify VEGF inhibitors. In general, such methods can include (a) designing an assay to measure the binding of a VEGF polypeptide to a VEGF receptor and (b) screening for compounds that disrupt this interaction. Examples of such assays are described elsewhere (e.g., U.S. Patent Application Publication No. 20020064779A1; Gustafsdottir et al., Clin. Chem., 54(7):1218-1225 (2008); and Peterson et al., Anal. Biochem., 378:8-14 (2008)). Once a compound is identified as being a candidate for disrupting the interaction of VEGF and VEGF receptors, the compound can be put through a secondary screen in which its ability to disrupt VEGF/VEGF receptor binding is determined in cells. Compounds that disrupt VEGF/VEGF receptor binding in cells can be further screened for the ability to inhibit venous stenosis formation as described herein.

In some cases, one or more VEGF inhibitors can be formulated into a pharmaceutically acceptable composition for delivery to the adventitia of a vein of an arteriovenous fistula or graft from a position outside the vein. For example, a therapeutically effective amount of an anti-VEGF antibody can be formulated together with one or more pharmaceutically acceptable carriers (additives) and/or diluents. A pharmaceutical composition can be formulated for administration in solid or liquid form including, without limitation, sterile solutions, suspensions, sustained-release formulations, powders, and granules.

In some cases, the methods and materials provided herein can be used to continuously infuse a composition having one or more VEGF inhibitors to the external surface of the vein of an AV graft. In some cases, a chronic delivery could be achieved by placing a composition having one or more VEGF inhibitors into a polymer that can be placed at the graft or fistula or graft site to elute the VEGF inhibitor(s) for an extended period of time. For example, a composition containing one or more VEGF inhibitors can be formulated into a sustained release device that can be positioned outside the vein to be treated such that the one or more VEGF inhibitors can be released and contact the adventitia of the vein of an arteriovenous fistula or graft, thereby reducing venous stenosis formation. In some cases, a sustained release device composed of chitosan, alginates, polyethylene glycol (PEG), poly lactic acid (PLA), copoly lactic acid/glycolic acid (PLGA), dextrans, acrylates, cyclodextrins, caprolactones, block copolymers, nanoparticles, or combinations thereof can be loaded with one or more VEGF inhibitors and implanted adjacent to or around the outside of a vein to be treated. In some cases, a pump (e.g., an implantable pump) can be used to deliver a composition containing one or more VEGF inhibitors to the adventitia of a vein to be treated. For example, a composition having one or more VEGF inhibitors can be delivered to an AV graft using a drug pump that can deliver a composition through a tube placed at the graft site or via a cuff wrapped around the graft.

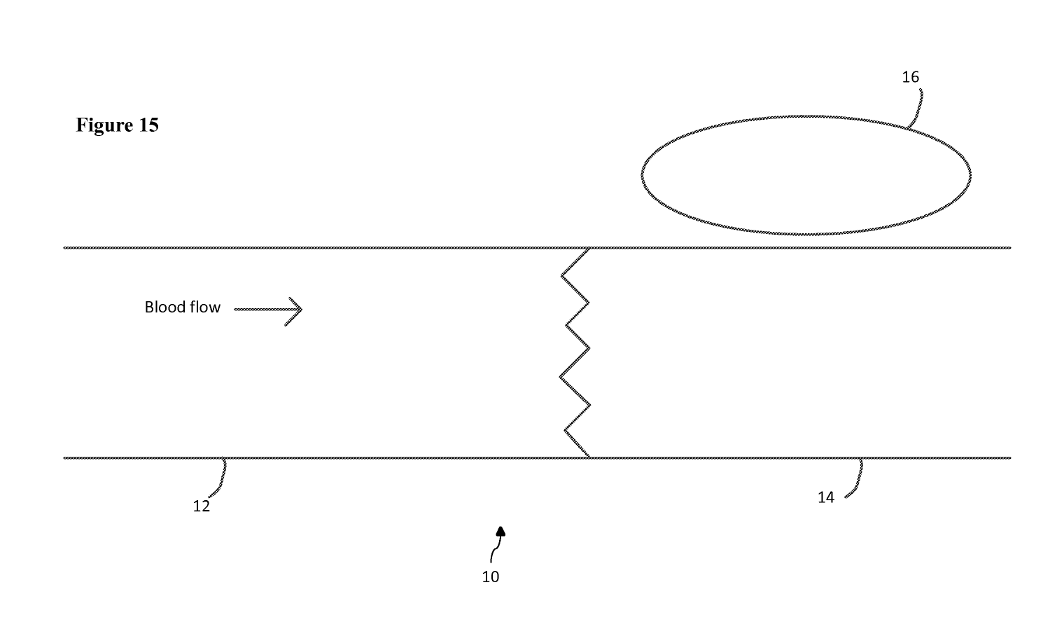

With reference to FIG. 15, an arteriovenous fistula or graft 10 can include artery 12 and vein 14. A sustained release device 16 can be implanted in a position outside of vein 14. For example, sustained release device 16 can be implanted adjacent to vein 14. Sustained release device 16 can be configured to release one or more VEGF inhibitors over extended periods of time (e.g., between one day and 24 months, between one week and 24 months, between one month and 24 months, between one week and 18 months, between one week and 12 months, between one day and 36 months, between one day and 48 months, between one month and 48 months, or between one week and 36 months). In some cases, sustained release device 16 can be configured such that additional VEGF inhibitor(s) can be added over time. For example, sustained release device 16 can be configured such that additional VEGF inhibitor(s) can be injected into sustained release device 16 when the initial amount of VEGF inhibitor(s) drops below a predetermined level.

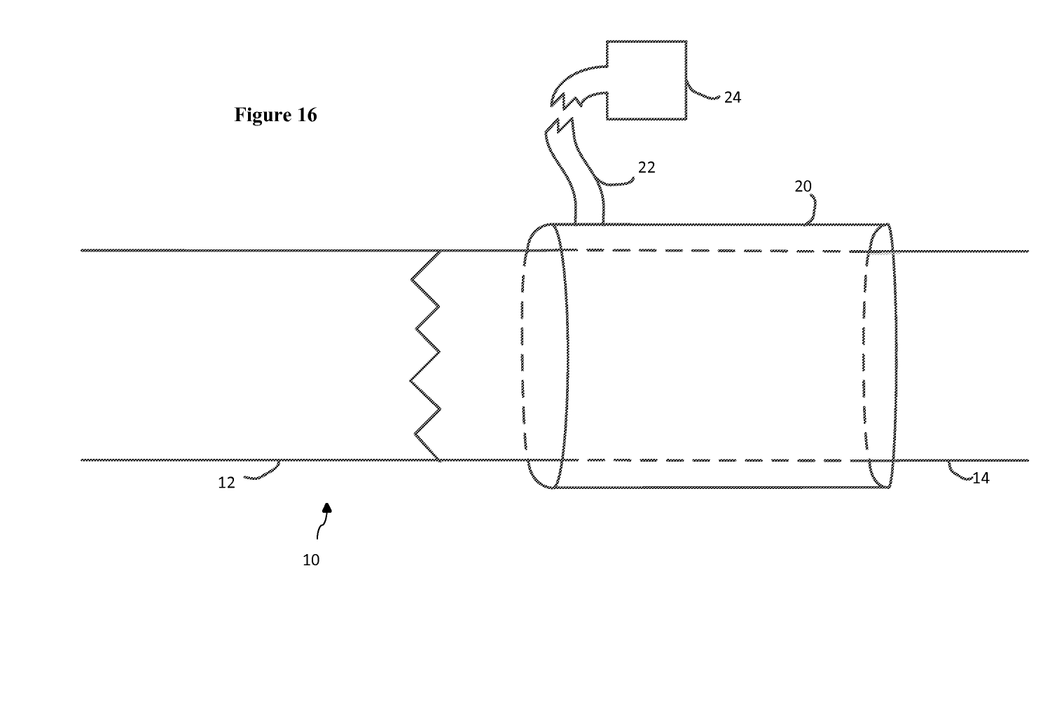

With reference to FIG. 16, a cuff device 20 can be implanted in a position at least partially around (e.g., completely around) vein 14. For example, cuff device 20 can be implanted to surround at least a segment of vein 14. Cuff device 20 can be configured to release one or more VEGF inhibitors over extended periods of time (e.g., between one day and 24 months, between one week and 24 months, between one month and 24 months, between one week and 18 months, between one week and 12 months, between one day and 36 months, between one day and 48 months, between one month and 48 months, or between one week and 36 months). In some cases, cuff device 20 can be attached to a pump 24 via a tubular member 22. Pump 24 can be configured to pump VEGF inhibitor(s) from a reservoir to cuff device 20. Cuff device 20 can include one or more outlets configured to allow the VEGF inhibitor(s) to be delivered to the adventitia of a vein to be treated.

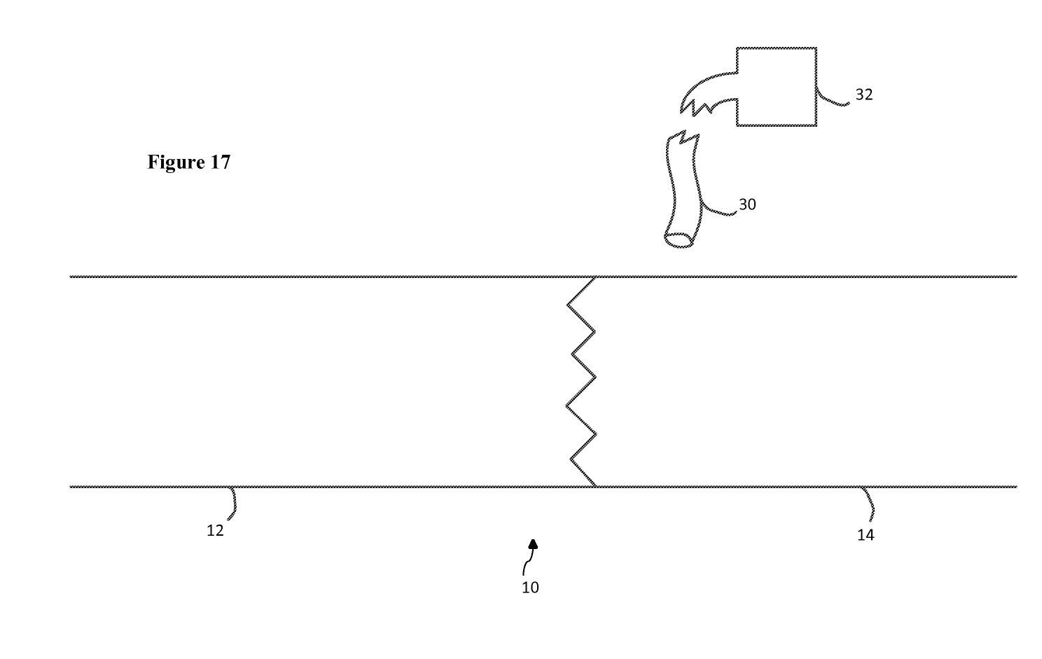

With reference to FIG. 17, a delivery device having a pump 32 and a tubular member 30 can be implanted at a position that allows a distal end opening of tubular member 30 to allow VEGF inhibitor(s) to be delivered to the adventitia of a vein to be treated. Pump 32 can be configured to pump VEGF inhibitor(s) from a reservoir to a distal end opening of tubular member 30.

A composition containing one or more VEGF inhibitors can be delivered to the adventitia of a vein to be treated in any amount, at any frequency, and for any duration effective to achieve a desired outcome (e.g., to reduce venous stenosis formation of an arteriovenous fistula or graft).

Effective doses can vary, as recognized by those skilled in the art, depending on the subject's propensity for venous stenosis formation, the route of delivery, the age and general health condition of the subject, excipient usage, the possibility of co-usage with other therapeutic treatments such as use of other agents, and the judgment of the treating physician.

An effective amount of a composition containing one or more VEGF inhibitors can be any amount that reduces venous stenosis formation without producing significant toxicity to the mammal. For example, an effective amount of a VEGF inhibitor such as an anti-VEGF antibody (e.g., Avastin) can be from about 5 mg/kg/day to about 15 mg/kg/day. In some cases, between about 50 mg and 200 mg of a VEGF inhibitor such as thalidomide can be administered to an average sized human (e.g., about 70 kg human) once a week in a slow release formulation. For example, about 800 mg of a VEGF inhibitor such as pazopanib can be administered to an average sized human (e.g., about 70 kg human) once a week. If a particular mammal fails to respond to a particular amount, then the amount of VEGF inhibitor can be increased by, for example, two fold. After receiving this higher amount, the mammal can be monitored for both responsiveness to the treatment and toxicity symptoms, and adjustments made accordingly. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, and route of deliver, may require an increase or decrease in the actual effective amount administered.

The frequency of administration can be any frequency that reduces venous stenosis formation without producing significant toxicity to the mammal. For example, the frequency of administration can be from about once a week to about three times a day, or from about twice a week to about four times a week, or from about once a day to about twice a day. The frequency of administration can remain constant or can be variable during the duration of treatment. A course of treatment with a composition containing one or more VEGF inhibitors can include rest periods. For example, a composition containing one or more VEGF inhibitors can be administered daily over a two month period followed by a two week rest period, and such a regimen can be repeated multiple times. As with the effective amount, various factors can influence the actual frequency of administration used for a particular application. For example, the effective amount, duration of treatment, use of multiple treatment agents, and route of delivery may require an increase or decrease in administration frequency.

An effective duration for administering a composition containing one or more VEGF inhibitors can be any duration that reduces venous stenosis formation without producing significant toxicity to the mammal. Thus, the effective duration can vary from several weeks to several months or years. In general, the effective duration for reducing venous stenosis formation can range in duration from several months to several years. In some cases, an effective duration can be for as long as an individual mammal is alive. Multiple factors can influence the actual effective duration used for a particular treatment. For example, an effective duration can vary with the frequency of administration, effective amount, use of multiple treatment agents, and route of delivery.

In certain instances, a course of treatment and the possible formation of venous stenosis can be monitored. Any method can be used to determine whether or not venous stenosis formation is reduced. For example, ultrasound, intravascular ultrasound, angiogram, computed tomographic analysis, or magnetic resonance angiography can be used to assess possible venous stenosis formation.

The invention will be further described in the following examples, which do not limit the scope of the invention described in the claims.

### EXAMPLES

### Example 1

### Inhibition of VEGF-A Reduces Venous Stenosis Formation in Arteriovenous Fistulas or Grafts

### Experimental Animals

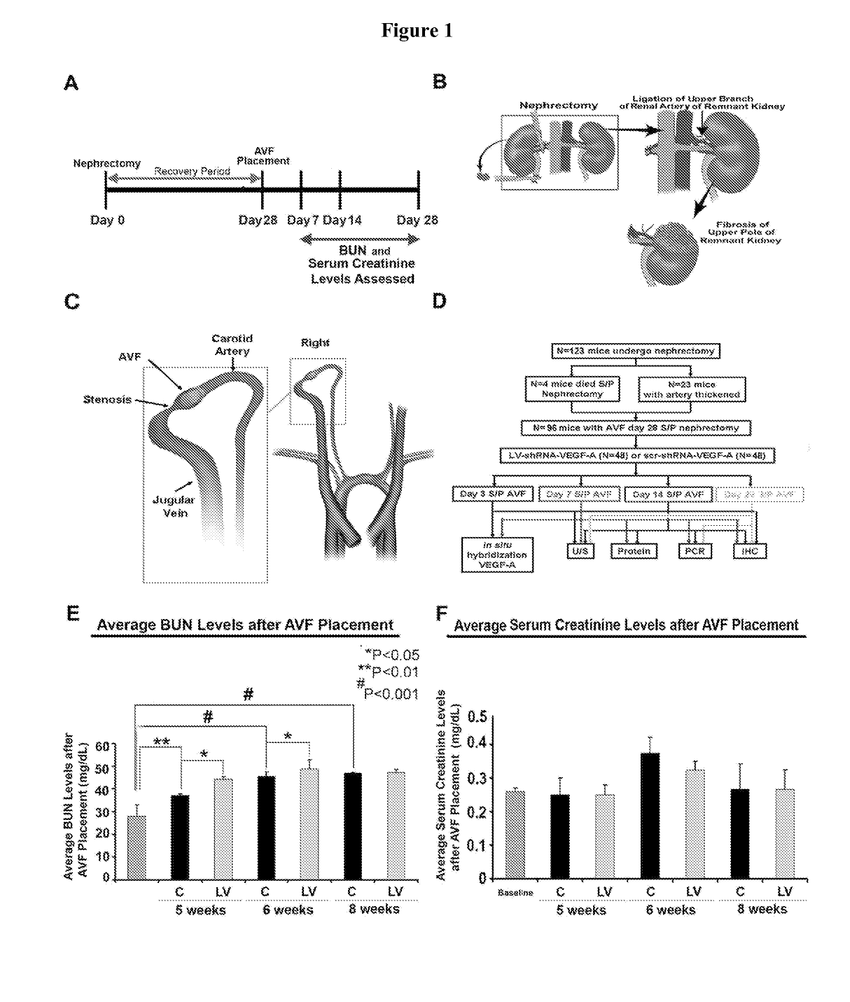

Appropriate Institutional Animal Care and Use Committee approval was obtained prior to performing any procedures. The housing and handling of the animals was performed in accordance with the Public Health Service Policy on Humane Care and Use of Laboratory Animals revised in 2000. Animals were housed at 22° C. temperature, 41% relative humidity, and 12-/12-hour light/dark cycles. Animals were allowed access to water and food ad libitum. Anesthesia was achieved with intraperitoneal injection of a mixture of ketamine hydrochloride (0.20 mg/g) and xylazine (0.02 mg/g) and maintained with intraperitoneal pentobarbital (20-40 mg/kg). One hundred and twenty three, male C57BL/6 mice (Jackson Laboratories, Bar Harbor, Me.) weighing 25-30 grams were used for the present study (FIGS. 1A-F). Chronic renal insufficiency was created by surgical removal of the right kidney accompanied by ligation of the arterial blood supply to the upper pole of the left kidney as described elsewhere (Misra et al., J. Vasc. Interv. Radiol., 21(8):1255-61 (2010); FIGS. 1A and 1B).

Four weeks after nephrectomy, an AVF was created by connecting the right carotid artery to the ipsilateral jugular vein (Misra et al., J. Vasc. Interv. Radiol., 21(8):1255-61 (2010) and Yang et al., J. Vasc. Interv. Radiol., 20:946-950 (2009); FIG. 1C). Five million particle forming units (PFU) of either lentivirus-shRNA-VEGF-A (LV-shRNA-VEGF-A) or scrambled-shRNA-VEGF-A (control shRNA, non targeting empty vector) in 10 μL of PBS were injected using a 30-guage needle, into the adventitia of the proximal outflow vein at the time of AVF creation (Turunen et al., Circ. Res., 105:604-609 (2009)). Animals were sacrificed at day 3 (D3), day 7 (D7), day 14 (D14), and day 28 (D28) following AV fistula or graft placement. Real time polymerase chain reaction (RT-PCR), protein, and histologic analyses were obtained (FIG. 1D). Serum BUN and creatinine were measured by removing blood from the tail vein at baseline (before nephrectomy), at AV-fistula or graft creation, and at the time of sacrifice.

### Vector Constructs

The shRNA for VEGF-A was obtained from Open Biosystems (Huntsville, Ala.; www.openbiosystems.com, RMM4534-NM_{—}001025250). For lentivirus preparation, 293T cells were seeded at a density of 6×10⁶/100-mm plate 24 hours before transfection. Cells were transfected with 2 μg of pLK0.1-puro-VEGF-A DNA using Effectene™ transfection reagent (Qiagen, Valencia, Calif.) according to the manufacturer's protocol. The medium was changed after 16 hours. Lentivirus was isolated 48 hours after transfection and used immediately for infection or was frozen at −80° C. for subsequent use. The plaque forming unit values for LV-shRNA-VEGF-A or control shRNA were calculated by subjecting different dilutions of lentivirus aliquots to QuickTiter™ Lentivirus Quantification Kit (Cell Biolabs, Inc. San Diego, Calif.) in accordance with manufacturer's instructions.

### Cell Culture

To determine the efficacy and efficiency of lentiviral silencing on VEGF-A expression, murine fibroblasts (AKR-2B) cells were seeded at 1×10⁶ cells in 100-mm plate for 24 hours prior to infection. One mL of lentivirus solution (about 2×10⁷ plaque-forming units/mL) and 5 mL of fresh medium were added to the AKR-2B cells with 10 μg/mL polybrene. The medium was changed after 16 hours, and AKR-2B colonies silenced for VEGF-A were selected with puromycin (1 μg/mL) for 48 hours before being analyzed by RT-PCR or Western blotting.

### Hypoxia Chamber

AKR-2B transfected with LV-shRNA-VEGF-A or control shRNA were made hypoxic for 24 or 72 hours as described elsewhere (Misra et al., J. Vasc. Interv. Radiol., 21:896-902 (2010)).

### Tissue Harvesting

At euthanasia, all mice were anesthetized, and the fistula or graft was dissected free of the surrounding tissue. Animals were euthanized by CO₂ asphyxiation, and the outflow veins harvested for RT-PCR, histologic, or protein analyses.

RNA Isolation

The tissue was stored in RNA stabilizing reagent (Qiagen; Gaithersburg, Md.) as per the manufacture's guidelines. To isolate the RNA, the specimens were homogenized, and total RNA isolated using RNeasy mini kit (Qiagen) (Misra et al., J. Vasc. Interv. Radiol., 21(8):1255-61 (2010) and Yang et al., J. Vasc. Interv. Radiol., 20:946-950 (2009)).

### Real Time Polymerase Chain Reaction (RT-PCR) Analysis

Expression for the gene of interest was determined using RT-PCR analysis (Yang et al., J. Vasc. Interv. Radiol., 20:946-950 (2009)). Briefly, first-strand complementary DNA (cDNA) was synthesized using superscript III first strand (Invitrogen; Carlsbad, Calif.) according to the manufacturer's guidelines. cDNAs specific for the genes analyzed were amplified using primers (Table 1). PCR products were analyzed on 1.5% (w/v) agarose gels containing 0.5-μg/mL ethidium bromide. Bands were quantified by scanning densitometry (Image J version 1.43; NIH; Bethesda, Md.). An area of the gel image that was devoid of signal was assigned to be the background value. Then each band representing the gene of interest was analyzed for the density above background. Next, it was normalized to the amount of loading of mRNA to 18S gene to ensure that there were no differences in loading and then pooled for all the animals in the different treatment groups for each time period.

TABLE 1

| | | Amplicon | |
|---|---|---|---|
| Gene | Sequence | Length | Cycles |
| HIF-1a | 5′-agtgatgaaagaattact-3′ (sense; SEQ ID NO: 1) | 2759 | 35 |
| | 5′-aataataccacttacaaca-3′ (antisense; SEQ ID NO: 2) | | |
| VEGF-A | 5′-atgaagtgatcaagttcatgg-3′ (sense; SEQ ID NO: 3) | 360 | 35 |
| | 5′-ggatcttggacaaacaaatgc-3′ (antis ens e; SEQ ID NO: 4) | | |
| VEGFR-1 | 5′-tttccatttgatactcttac-3′ (sense; SEQ ID NO: 5) | 310 | 35 |
| | 5′-tcttagttgctttaccaggg-3 ′ (antisense; SEQ ID NO: 6) | | |
| VEGFR-2 | 5′-tgtggttgtaggatataggat-3′ (sense; SEQ ID NO: 7) | 338 | 35 |
| | 5′-aaaggctttgtgtgaactcgg-3′ (antisense; SEQ ID NO: 8) | | |
| MMP-2 | 5′-agatcttcttcttcaaggaccggtt-3′ (sense; SEQ ID NO: 9) | 225 | 35 |
| | 5′-ggctggtcagtggcttggggta-3′(antisense; SEQ ID | | |
| | NO: 10) | | |
| MMP-9 | 5′-gtttttgatgctattgctgagatcca-3′ (sense; SEQ ID NO: 11) | 208 | 35 |
| | 5′-cccacatttgacgtccagagaagaa-3′(antisense; SEQ ID | | |
| | NO: 12) | | |
| TIMP-1 | 5′-ggcatcctcttgttgctatcactg-3′ (sense; SEQ ID NO: 13) | 169 | 35 |
| | 5′-gtcatcttgatctcatcccgctgg-3′ (antisense; SEQ ID | | |
| | NO: 14) | | |
| TIMP-2 | 5′-ctcgctggacgttggaggaaagaa-3′ (sense; SEQ ID | 155 | 35 |
| | NO: 15) | | |
| | 5′-agcccatctggtacctgtggttca-3′ (antisense; SEQ ID | | |
| | NO: 16) | | |
| ADAMTS-1 | 5′-cattaacggacaccctgctt-3′ (sense; SEQ ID NO: 17) | 166 | 35 |
| | 5′-cgtgggacacacatttcaag-3′ (antisense; SEQ ID NO: 18) | | |
| 18S | 5′-agctaggaataatggaatag-3′ (sense; SEQ ID NO: 19) | 150 | 19 |
| | 5′-aatcaagaacgaaagtcggag-3′(antisense; SEQ ID | | |
| | NO: 20) | | |

In situ hybridization for VEGF-A
In situ hybridization for VEGF-A was performed as described elsewhere (Basu et al., Nat. Med., 7:569-574 (2001)). Briefly, the digoxigenin (DIG) labeled complementary RNA probe was made with plasmid pBS-164-VEGF (Gift from Andreas Nagy; Toronto, Canada) using DIG RNA labeling kit with T7 RNA polymerase for antisense (complementary to VEGF mRNA) probe and T3 RNA polymerase for sense (control) probe (Roche Applied Science, Indianapolis, Ind.). The probe hybridization was performed as per guidelines from Roche Applied Science. The probe hybridization was visualized by using anti-DIG-alkaline phosphatase antibody and NBT-BCIP solution as substrate (Roche Applied Science; Indianapolis, Ind.).

### SDS PAGE Zymography for MMP-2 and MMP-9

MMP-2 and MMP-9 polypeptide activities were determined using zymographic analysis. This was performed on homogenates from cultured cells or outflow veins transfected with either LV-shRNA-VEGF-A or control shRNA as described elsewhere (Misra et al., Kidney Int., 68:2890-2900 (2005) and Misra et al., Am. J. Physiol. Heart Circ. Physiol., 294:H2219-2230 (2008)).

### Western Blot of α-SMA

Differentiation of fibroblasts to myofibroblasts was assessed by performing Western blot analysis for α-SMA. The cultured cells were processed for Western analysis using rabbit polyclonal antibody as described elsewhere (Misra et al., Am. J. Physiol. Heart Circ. Physiol., 294:H2219-2230 (2008)).

### Caspase 3 Activity

Apoptosis was assessed using an ELISA assay for caspase 3. Cellular polypeptides were extracted from cultured cells and mouse tissue. The enzymatic activity of caspase 3 was accessed by Caspase Glo assay (G811C; Promega, Madison, Wis.).

### Proliferation Assay

AKR-2B transfected with LV-shRNA-VEGF-A or control shRNA at 20,000 cells were seeded in 24-well plates and cultured for 24, 48, and 72 hours in DMEM medium. After 20, 44, and 68 hours, 1 mCi of (³H) thymidine was added to each well. Four hours later, the cells were washed with chilled PBS, fixed with 100% cold methanol, and collected for measurement of trichloroacetic acid-precipitable for radioactivity. Experiments were repeated at least three times for each time point.

### Invasion and Cell Migration Assay

AKR-2B transfected with LV-shRNA-VEGF-A or control shRNA at 5000 cells were seeded in 8-micron trans-wells, pre-coated with low growth factor matrigel in a serum free media. The complete media was supplemented under the trans-well and incubated for 6 hours at 37° C. After 6 hours, trans-wells were washed with PBS and fixed with paraformaldehye (4% v/v). Finally, trans-wells were stained with bromophenol (0.1%) solution. The cells from the upper side were removed with cotton tip applicators. The cells at the bottom side were counted for analysis.

### Immunohistochemistry

Cellular proliferation was determined by staining for Ki-67 on sections removed from the outflow vein by performing quantification at different time points. Smooth muscle density was determined by staining for α-SMA on sections removed from the outflow vein by performing quantification at the different time points Immunohistochemistry for Ki-67 and α-SMA were performed on paraffin-embedded sections from the outflow vein after transfection with either LV-shRNA-VEGF-A or control shRNA using the Vectastain Elite ABC system (Vector Laboratories; Burlingame, Calif.) as described elsewhere (Misra et al., Kidney Int., 68:2890-2900 (2005) and Misra et al., Am. J. Physiol. Heart Circ. Physiol., 294:H2219-2230 (2008)). The following antibodies were used: mouse monoclonal antibody Ki-67 (DAKO; Carpentaria, Calif.; 1:400) or rabbit polyclonal antibody to mouse for α-SMA (Abcam; Cambridge, Mass.; 1:400).

Hypoxyprobe Staining at Day 14 and 28 at LV-shRNA-VEGF-A and Scrambled shRNA VEGF-A Transfected Vessels

Hypoxic changes in the outflow vein after transfection with either LV-shRNA-VEGF-A or control shRNA were assessed using hypoxyprobe (Hypoxyprobe™-1, a substituted derivative of pimonidazole hydrochloride). Hypoxyprobe™-1, upon activation, forms stable covalent adducts with thiol groups of proteins, peptides and amino acids of hypoxic tissue. Mice were injected with 60 mg/kg Hypoxyprobe™-1 i.p. (EMD Millipore; Billerica, Mass.). Thirty minutes following injection, mice were sacrificed, and outflow veins were dissected and fixed as specified for histological analysis. Four-micrometer paraffin embedded sections were stained with the anti-hypoxyprobe-1 antibody as per the manufacturer's directions.

### TUNEL Staining at Day 14 and 28

TUNEL staining was performed on paraffin-embedded sections from the outflow vein after transfection with either LV-shRNA-VEGF-A or control shRNA as specified by the manufacturer (DeadEnd Colorimetric tunnel assay system, G7360; Promega).

### Morphometry and Image Analysis

Sections immunostained for hematoxylin and eosin stains were viewed with an Axioplan 2 Microscope (Zeiss; Oberkochen, Germany) equipped with a Neo-Fluor×20/0.50 objective and digitized to capture a minimum of 3090×3900 pixels using a Axiocam camera (Zeiss) (Misra et al., Kidney Int., 68:2890-2900 (2005) and Misra et al., Am. J. Physiol. Heart Circ. Physiol., 294:H2219-2230 (2008)). Images covering one entire cross-section from each section of the outflow vein transfected with either LV-shRNA-VEGF-A or control shRNA were acquired and analyzed using KS 400 Image Analysis software (Zeiss). Ki-67 (stained brown), α-SMA positive (stained pink), TUNEL positive (stained brown), or in situ hybridization positive (stained brown) were highlighted, in turn, by selecting the appropriate RGB (red-green-blue) color intensity range and then counted. The color intensity was adjusted for each section to account for decreasing intensity of positive staining over time. This was repeated twice to ensure intraobserver variability was less than 10%. Sections were subsequently viewed with an Axioplan 2 Microscope (Zeiss) equipped with a Neo-Fluor×20/0.50 objective and digitized to capture at least 1030×1300 pixels, and cell density determined along with the vessel wall and luminal vessel areas. The area was measured by tracing the vessel wall using an automated program (Misra et al., Am. J. Physiol. Heart Circ. Physiol., 294:H2219-2230 (2008)).

Ultrasound Measurements of the Outflow Vein after Transfection with Either LV-shRNA-VEGF-A or Control shRNA at Day 3, 7, 14, and 28

Ultrasound was used to assess the post-operative function of the AVF. Ultrasound of the outflow vein after transfection with either LV-shRNA-VEGF-A or control shRNA was performed to assess for blood velocity, diameter, and patency in 20 animals after the placement of the fistula or graft at day 3, 7, 14, and 28 as described elsewhere (Yang et al., J. Vasc. Interv. Radiol., 20:946-950 (2009)). Measurements and analysis were performed with a Vevo 770 High-Resolution In Vivo Micro-Imaging System (VisualSonics Inc.; Toronto, Ontario, Canada) using an RMV708 transducer capable of up to 240 frames per second, frequencies from 22 to 83 MHz (minimum 30-micron resolution), and a fixed focal depth of 4.5-mm. Doppler analysis was performed with the angle between the incident sound beam and the blood flow less than 80°, as specified by VisualSonics for use of their system. The cuff used for making the anastomosis is visible on ultrasound and was used as a reference to identify the outflow vein. Just distal to the cuff, measurements for the outflow vein were made.

### Statistical Methods

Data were expressed as mean±SEM. Two groups were compared with 2-tailed unpaired Student's t-test and more than 2 groups with 1-way ANOVA followed by Neuman-Keuls multiple comparison test. Spearman's rank coefficient was used for correlation coefficient. Significant difference from control value was indicated by *P<0.05, **P<0.01, or #P<0.001. SAS version 9 (SAS Institute Inc., Cary, N.C.) was used for statistical analyses.

### Results

### Surgical Outcomes

One hundred and twenty three male C57BL/6 mice weighing 25-30 grams underwent right nephrectomy and left upper pole occlusion surgery (FIG. 1B). Four mice died after nephrectomy, and twenty-three had significant arterial thickening and inflammation such that a new AV fistula or graft could not be placed. Ninety-six mice remained, and they comprised the animals reported in this example. The mice underwent placement of an AVF to connect the right carotid artery to the ipsilateral jugular vein (FIG. 1C). Next, either 1×10⁶ PFU of LV-shRNA-VEGF-A (LV, n=48) or scrambled-shRNA-VEGF-A (control, C, n=48) was injected into the adventitia of the outflow vein where the stenosis forms in this model (Yang et al., J. Vasc. Interv. Radiol., 20:946-950 (2009) and Misra et al., J. Vasc. Interv. Radiol., 21:1255-1261 (2010)). Animals were sacrificed for gene expression, protein, or histologic analyses at day 3 (D3), 7 (D7), 14 (D14), and 28 (D28) after AVF placement (FIG. 1D).

Serum BUN and Creatinine after Nephrectomy

In this model, elevated creatinine and BUN levels similar to what is observed in the typical clinical scenario were observed. The mean BUN and creatinine at baseline was 28±5 mg/dL and 0.26±0.1 mg/dL, respectively, and increased significantly at 1, 5, 6, and 8 weeks after nephrectomy (FIGS. 1E and 1F, P<0.05).

### Adventitial Transfection of LV-shRNA-VEGF-A to the Outflow Vein Reduces Gene Expression of VEGF-A at Days 3, 7, and 14

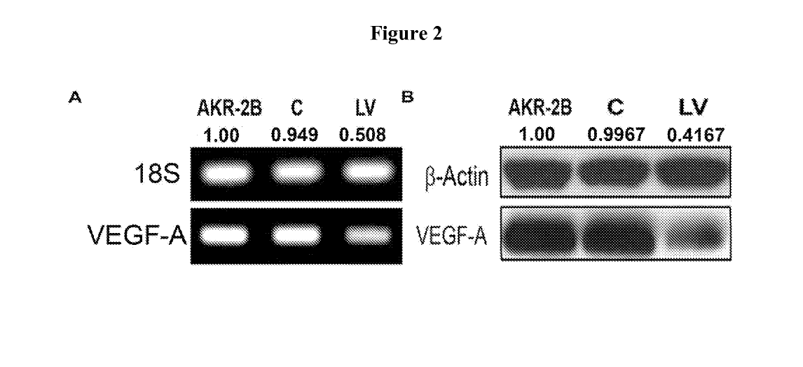

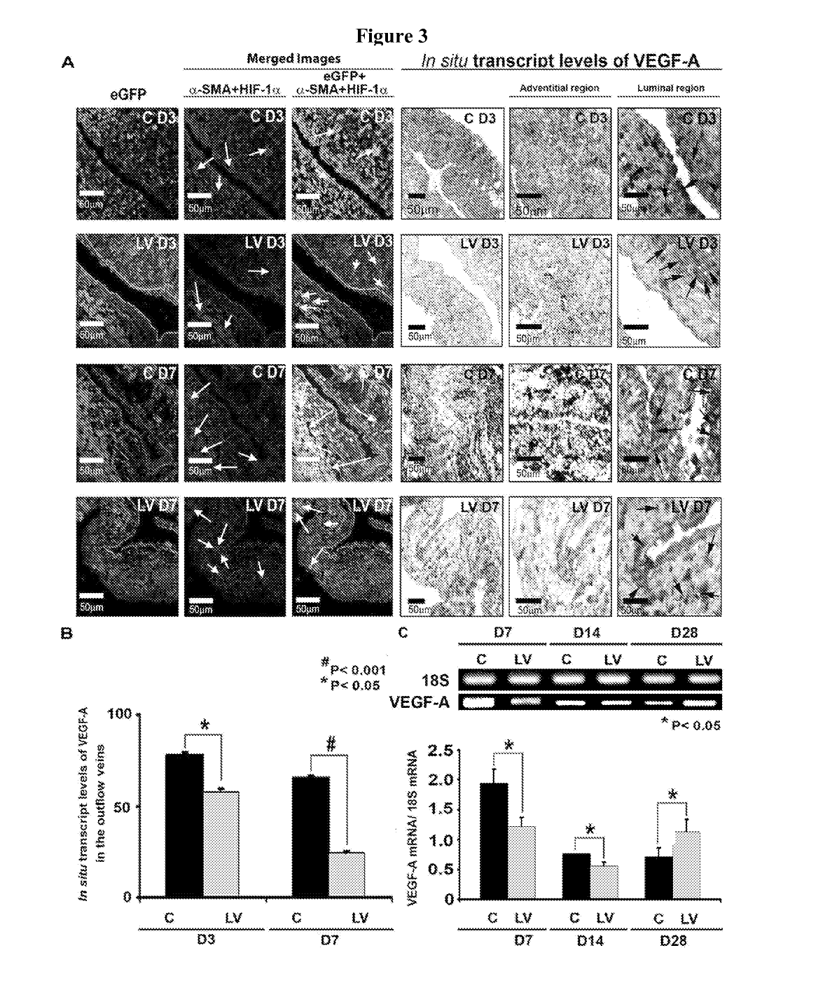

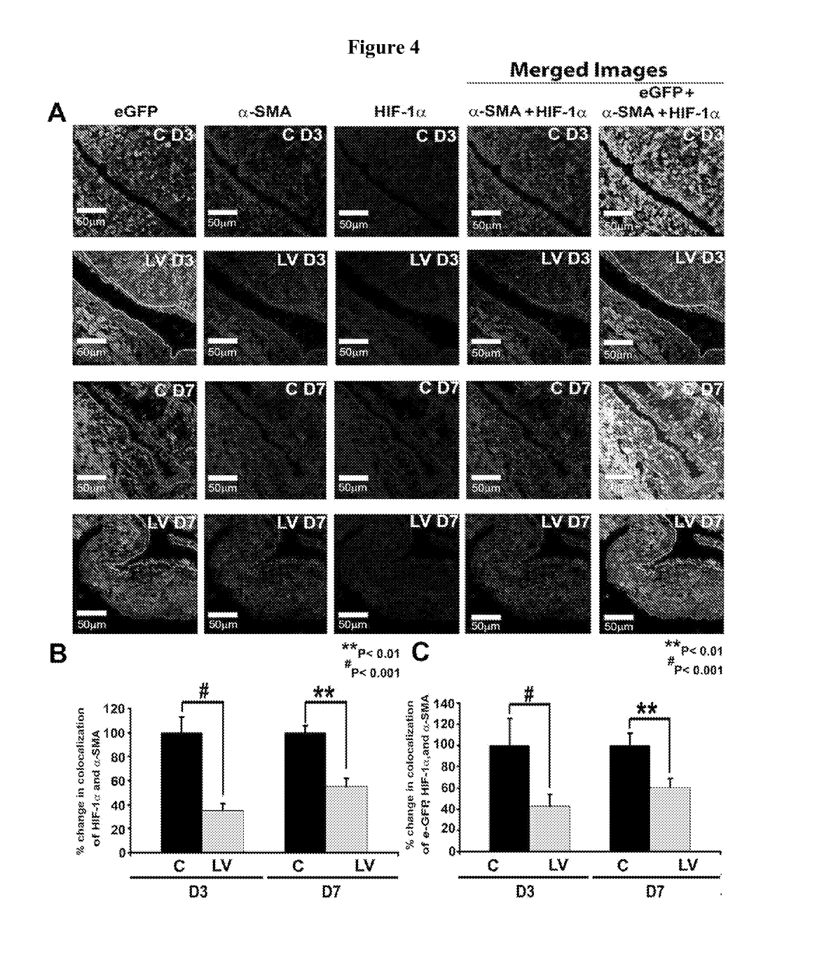

The efficacy of reducing VEGF-A gene expression in vitro in AKR-2B (murine fibroblast cell line) cells transfected with either LV-shRNA-VEGF-A (LV) or scrambled shRNA-VEGF-A (C, control) was determined using both RT-PCR (FIG. 2A) and Western blot analyses (FIG. 2B) with greater than two fold decrease in VEGF-A expression in the LV-shRNA-VEGF-A transfected cells when compared to controls. To ascertain, if similar findings are present in vivo, experiments were designed and performed to determine the distribution of the lentivirus in the vasculature after delivery to the vessel wall. Lentivirus was delivered using a GFP tagged control and shRNA-VEGF-A lentivirus. Using confocal microscopy, the results demonstrate that the GFP tagged control was distributed evenly throughout the vessel with expression persisting for seven days following transfection (FIG. 3A, first column) Confocal microscopy for cells staining positive for both α-SMA and HIF-1α (pink) revealed that there was a decrease in the expression in the LV-shRNA-VEGF-A transfected vessels when compared to controls (FIG. 3A, second and third column) Semiquantitative analysis revealed a significant reduction in the cells staining positive for both HIF-1α and α-SMA and also cells staining positive for eGFP, HIF-1α, and α-SMA at both days 3 and 7 (FIGS. 4B and 4C).

The amount of reduction and localization of VEGF-A gene expression was determined in vivo using in situ hybridization for VEGF-A. By day 3, there was a reduction of mRNA for VEGF-A being localized to the media and adventitia and by day 7, it was localized to the media and intima (FIG. 3A, fourth to sixth column). In contrast, the vessels transfected with control shRNA exhibited increased mRNA expression of VEGF-A in the adventitia and media by day 3, and in the media and intima by day 7. Semiquantitative analysis of the in situ hybridization was performed and confirmed a significant reduction in the mRNA levels in the LV-shRNA-VEGF-A transfected vessels when compared to control vessels at both day 3 (58±2.6 vs. 78±3.2, respectively, P<0.05, Average reduction: 26%) and day 7 (24.5±3.3 vs. 65.3±6, respectively, P<0.001, Average reduction: 62%, FIG. 1B).

The next set of studies used RT-PCR analysis for VEGF-A on sections removed from the outflow vein at days 7, 14, and 28 after lentiviral transfection. By day 7, the mean gene expression of VEGF-A at the LV-shRNA-VEGF-A transfected vessels was significantly lower than the control vessels (1.1±0.25 vs. 1.96±0.25, respectively, P<0.05, Average reduction: 44%, FIG. 3C), and by day 14, it remained significantly lower in the LV-shRNA-VEGF-A transfected vessels when compared to controls (0.56±0.07 vs. 0.75±0.02, respectively, P<0.05, Average reduction: 25%). By day 28, there was recovery of the VEGF-A mRNA levels in the LV-shRNA-VEGF-A transfected specimens with a significant increase when compared to controls shRNA (1.1±0.23 vs. 0.53±0.06, respectively, P<0.05, Average increase: 207%). There was a strong correlation between mRNA expression of VEGF-A with length of time of fistula or graft placement for both the LV-shRNA-VEGF-A and control shRNA transfected vessels by both in situ hybridization and RT-PCR analyses for days 3, 7, and 14 (Spearman rank: r=1). Taken collectively, these results indicate that mRNA levels of VEGF-A can be reduced at the outflow vein using adventitial delivery of LV-shRNA-VEGF-A, and the reduction in VEGF-A mRNA signal lasts for 2-weeks after delivery. This, however, increases by 4-weeks in the LV-shRNA-VEGF-A transfected vessels when compared to controls. In addition, the reduction in mRNA was reduced at adventitia and media at day 3 (brown staining in cells), and by day 7, it was in the media and intima following a “top down effect.”

### Adventitial Transfection of LV-shRNA-VEGF-A to the Outflow Vein Promotes Positive Vascular Remodeling at Days 3, 7, and 14

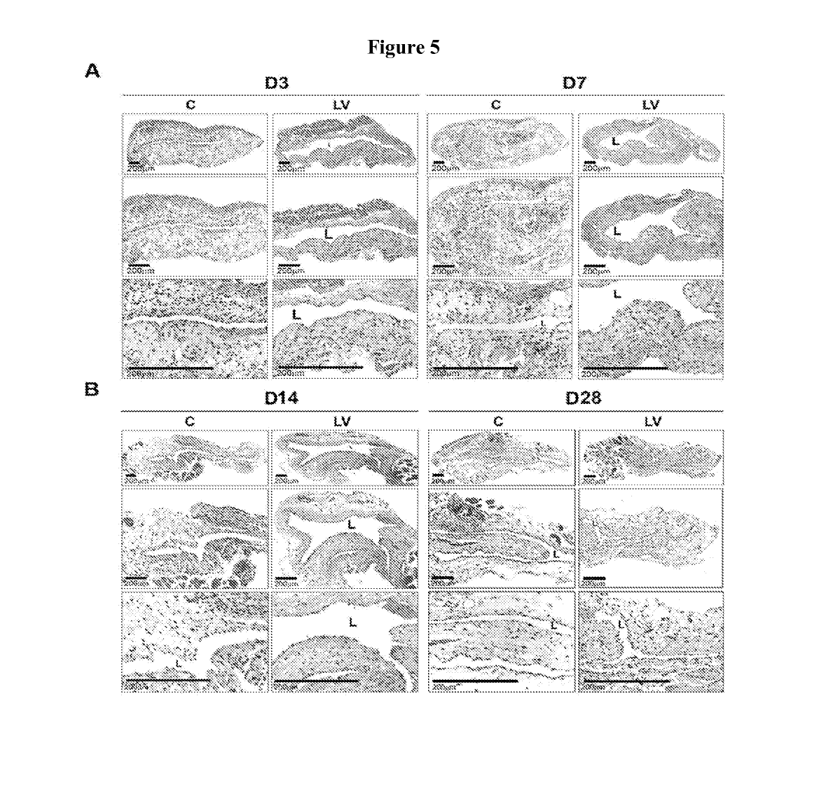

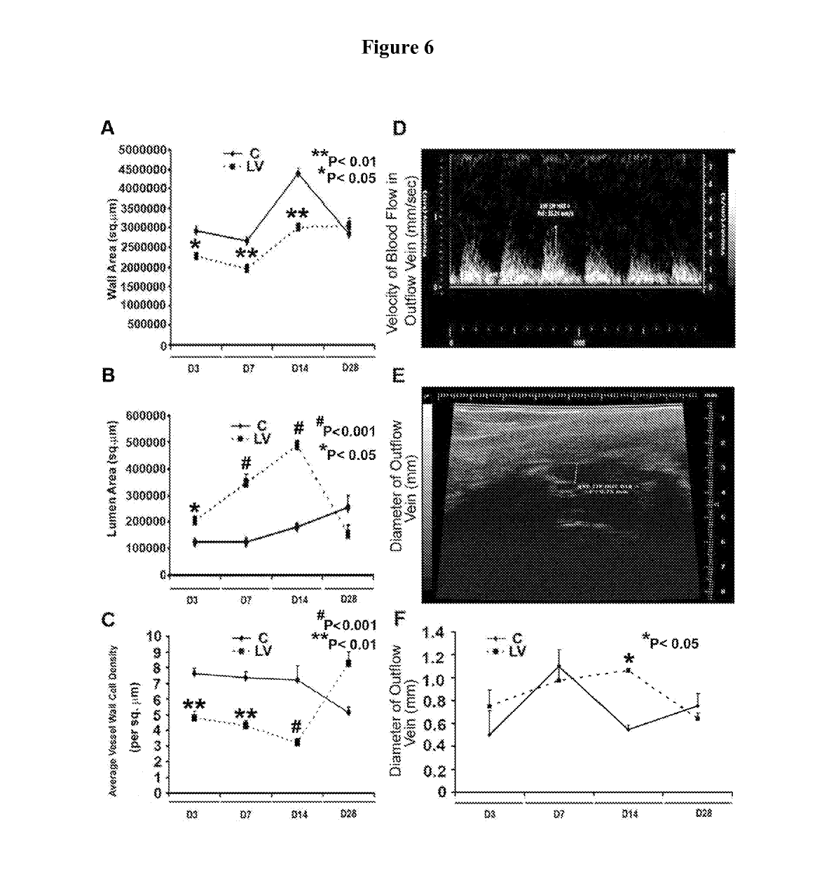

It was hypothesized that reducing mRNA levels of VEGF-A by adventitial transfection of LV-shRNA-VEGF-A to the outflow vein would result in a reduction of area of the vessel wall and the cellular density while increasing lumen area (FIGS. 5A and 5B). At days 3-14, there was a significant decrease in the average wall area (FIG. 6A) in the LV-shRNA-VEGF-A transfected vessels when compared to the controls (P<0.05 for day 3 and P<0.01 for days 7 and 14) with an average reduction of 40% by day 14. By day 28, the average wall area in the LV-shRNA-VEGF-A transfected vessels increased and was similar to control vessels (P=NS). The average wall area correlated strongly with length of time of the fistula or graft placement (day 3, 7, 14, or 28) for both the control shRNA group (Spearman rank: r=0.8) and LV-shRNA-VEGF-A (Spearman rank: r=0.8). At days 3-14, the average lumen area of the outflow vein was found to be significantly higher in the LV-shRNA-VEGF-A transfected vessels when compared to the control vessels (P<0.05 for day 3, P<0.001 for days 7-14, FIG. 6B) with an average increase of 500% by day 14. The average lumen area correlated strongly with the length of time for the fistula or graft for the control shRNA group (Spearman rank: r=0.8) and strongly correlated for LV-shRNA-VEGF-A (Spearman rank: r=1 until day 14, and weak correlation until day 28).

The following was performed to determine if the decrease in wall area with increase in the lumen vessel area in the LV-shRNA-VEGF-A group was due to a decrease in cell density at the outflow vein. Quantitative analysis for average cell density was performed in the LV-shRNA-VEGF-A or control vessels at days 3, 7, 14, and 28 after AVF placement. By days 3-14, the cell density of the LV-shRNA-VEGF-A transfected vessels was significantly less than that of the control vessels (P<0.01 for days 3 and 7, P<0.001 for day 14) with an average reduction of 60% by day 14. By day 28, the cell density had increased and was significantly higher in the LV-shRNA-VEGF-A transfected vessels when compared to controls (P<0.001). There was a strong correlation between cellular density with the length of time of fistula or graft placement for the LV-shRNA-VEGF-A transfected vessels (Spearman rank: r=0.86) and a moderate correlation with the control shRNA group (Spearman rank: r=0.55) (FIG. 6C).

Ultrasound of the Outflow Vein of the LV-shRNA-VEGF-A and Control shRNA Transfected Vessels at Days 3, 7, 14, and 28

Clinically, ultrasound is used to assess the patency and function of AVFs following surgical placement (Singh et al., Radiology, 246:299-305 (2008)). To assess the diameter of the outflow vein and vessel patency at day 3, 7, and 14 following AVF placement, ultrasound of the outflow vein after transfection with either LV-shRNA-VEGF-A or control shRNA was performed. A typical waveform of the blood velocity and measurement of AVF diameter are shown in FIGS. 6D and 6E). The diameter of the outflow vein in both groups was determined (FIG. 6F). The average diameter of the outflow vein of the LV-shRNA-VEGF-A transfected vessels increased steadily over time, implying that the lumen vessel area is increasing as well. This is consistent with the histomorphometric analysis performed in a separate group of animals. By day 14, the average diameter of the outflow vein of the LV-shRNA-VEGF-A transfected vessels was significantly higher than the controls (1.2±0.03 vs. 0.6±0.02, respectively, P<0.05, Average increase: 200%), and by day 28, it was the same in both groups (P=NS). The diameter of the outflow vein correlated strongly with length of time of fistula or graft placement for the LV-shRNA-VEGF-A group (Spearman rank: r=0.969) and correlated weakly for control shRNA (Spearman rank: r=0.07).

The patency of the AVF was assessed by ultrasound, which demonstrated that by day 3, there was 100% patency in the LV-shRNA-VEGF-A transfected vessels (n=2) when compared to 50% for the controls (n=2). By day 7, this decreased to 80% (n=5) vs. 75% (n=4), by day 14, 50% (n=6) vs. 33% (n=3), and by day 28, it was 20% (n=5) vs. 0% (n=5) (LV-shRNA-VEGF-A transfected vessels vs. controls, respectively). Although not statistically significant, the LV-shRNA-VEGF-A transfected vessels of the AVF had better patency rates at all time points when compared to the controls.

### Adventitial Transfection of LV-shRNA-VEGF-A to the Outflow Vein Increases Apoptosis at Day 3, 7, and 14

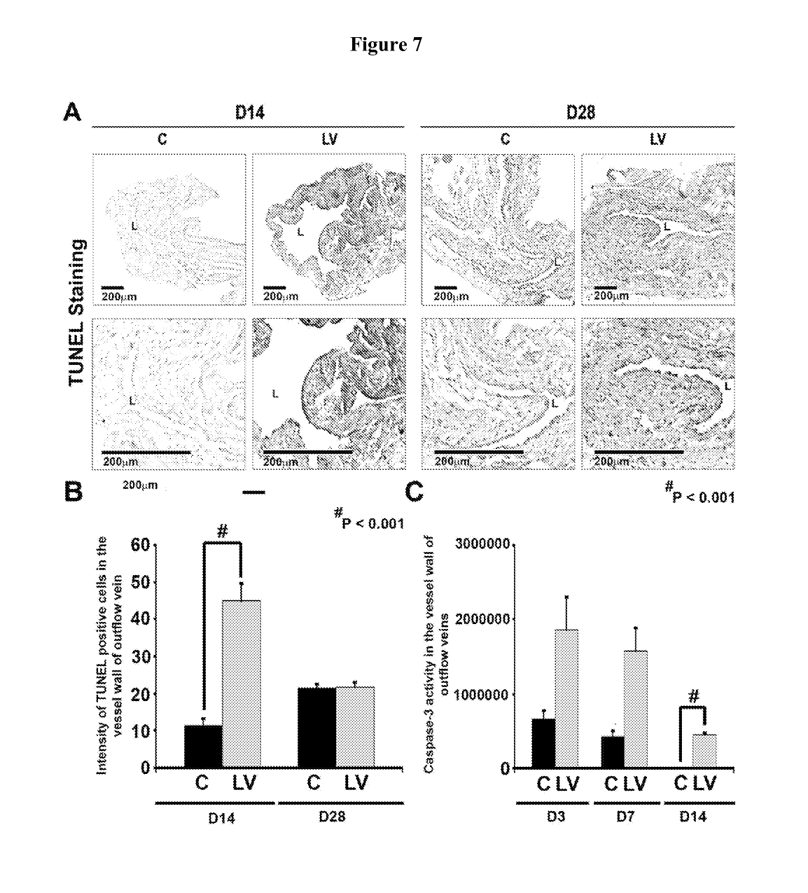

It was hypothesized that the decrease in cell density was due to an increase in apoptosis. Apoptosis was assessed by performing TUNEL staining in sections removed from the outflow vein at day 14 and 28 after transfection with either LV-shRNA-VEGF-A or control shRNA (FIGS. 7A and 7B). By day 14, the average intensity of cells staining positive for TUNEL (brown) at the outflow vein of the LV-shRNA-VEGF-A group was significantly higher than the control group (19.8±1.7 vs. 0.31±0.08, respectively, average increase: 640%, P<0.001, FIG. 7B). By day 28, the average intensity of the TUNEL staining in both groups was the same (P=NS). The average intensity of the TUNEL staining correlated strongly with length of time of fistula or graft placement for the LV-shRNA-VEGF-A and control shRNA groups (Spearman rank: r=1).

Caspase 3 is an effector of apoptosis. It was hypothesized that increased caspase 3 activity would be present in sections removed from outflow vein after transfection with either LV-shRNA-VEGF-A or control shRNA. By day 14, the average caspase 3 activity was significantly higher in the LV-shRNA-VEGF-A transfected vessels when compared to controls (average increase: 328%, P<0.001, FIG. 7C). The average caspase 3 activity correlated strongly with the length of time of fistula or graft placement for the LV-shRNA-VEGF-A and control shRNA groups (Spearman rank: r=1). Overall, these results indicate that adventitial delivery of LV-shRNA-VEGF-A results in a significant increase in caspase 3 activity and accompanying increased TUNEL staining at the outflow vein by day 14, strongly suggesting that the decrease in the cellular density of the LV-shRNA-VEGF-A transfected vessels is in part due to apoptosis.

### Adventitial Transfection of LV-shRNA-VEGF-A to the Outflow Vein Decreases Cellular Proliferation

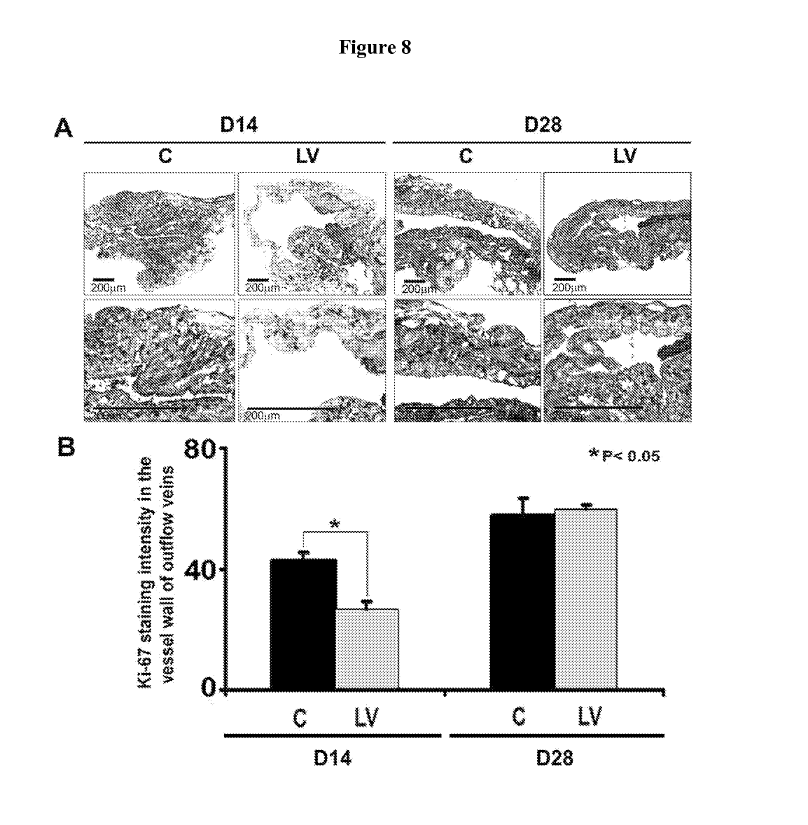

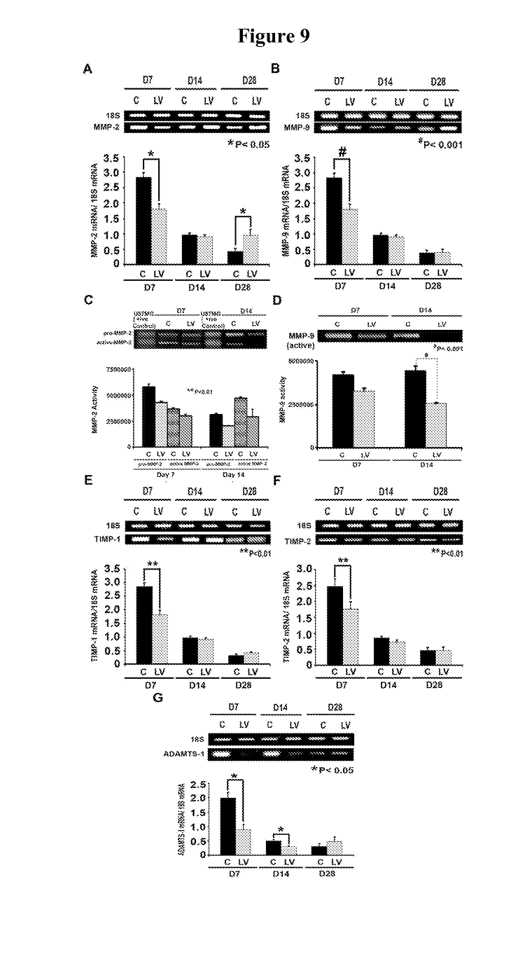

The following was performed to determine whether the decrease in cell density was due to a decrease in cell proliferation as demonstrated by Ki-67 staining on sections from the outflow vein after transfection with either LV-shRNA-VEGF-A or control shRNA (FIG. 8A). By day 14, the average intensity of cells staining positive for Ki-67 (brown) in the LV-shRNA-VEGF-A group was significantly lower than the control group (26.7±1.8 vs. 43.5±2.13, respectively, average reduction: 39%, P<0.05, FIG. 8B). By day 28, there was no difference in Ki-67 staining in the two groups (P=NS). The average intensity of the Ki-67 staining correlated strongly with length of time of fistula or graft placement for the LV-shRNA-VEGF-A and control shRNA groups (Spearman rank: r=1), suggesting cellular proliferation in the scrambled group is increasing with length of time of fistula or graft and decreasing in the LV-shRNA-VEGF-A group.

### Adventitial Transfection of LV-shRNA-VEGF-A to the Outflow Vein Reduces Expression of MMP-2, MMP-9, TIMP-1, and TIMP-2 at the Outflow Vein at Day 7

Several studies have shown that there is increased expression of MMP-2 and MMP-9 in animal models of hemodialysis AVF and graft failure and clinical samples. MMP-2 and MMP-9 gene products are thought to be responsible for cellular proliferation and cell migration resulting in VNH formation. To test this hypothesis, a set of experiments were designed to ascertain if reducing VEGF-A expression would lead to a reduction in MMP-2 and MMP-9 expression (Wang et al., Circ. Res., 83:832-840 (1998)). Gene expression of MMP-2, MMP-9, TIMP-1, and TIMP-2 was determined by RT-PCR analysis on specimens removed from the outflow vein transfected with either LV-shRNA-VEGF-A or control shRNA at day 7, 14, and 28. By day 7, the average gene expression of both MMP-2 and MMP-9 was significantly lower in the LV-shRNA-VEGF-A transfected vessels when compared to control shRNA (MMP-2: 1.96±0.26 vs. 2.91±0.29, respectively, P<0.05, Average reduction: 33%, FIG. 9A and MMP-9: 0.98±0.13 vs. 2.63±0.17, respectively, P<0.001, Average reduction: 63%, FIG. 9B). By day 14, there was no difference in the mean gene expression of MMP-2 and MMP-9 between both groups (P=NS), however, by day 28, there was a significant increase in MMP-2 expression at the LV-shRNA transfected vessels when compared to controls (0.96±0.2 vs. 0.43±0.1, respectively, P<0.05, Average increase: 225%). The average gene expression of MMP-2 and MMP-9 at the outflow vein correlated strongly with the length of time of fistula or graft placement for the LV-shRNA-VEGF-A and control shRNA groups (Spearman rank: r=1).

Because the translation of the protein lags behind the gene changes, the protein activity of MMP-2 and MMP-9 was assessed using zymography performed on sections from the outflow vein transfected with either LV-shRNA-VEGF-A or control shRNA at day 7 and 14. The average MMP-2 activity was decreased in the LV-shRNA-VEGF-A transfected vessels when compared to controls (P=NS, FIG. 9C). By day 14, the average MMP-9 activity was significantly lower in the LV-shRNA-VEGF-A transfected vessels when compared to control vessels (4411293±161838 vs. 2700581±1631901, respectively, P<0.001, Average reduction: 39%, FIG. 9D).

The following was performed to determine the gene expression of TIMP-1 and TIMP-2, which are inhibitors of MMP-2 and MMP-9. By day 7, the average gene expression of TIMP-1 and TIMP-2 was significantly lower in the LV-shRNA-VEGF-A transfected vessels when compared to control shRNA (TIMP-1: 1.69±0.14 vs. 2.88±0.18, respectively, P<0.05, Average reduction: 41%, FIG. 9E and TIMP-2: 1.72±0.19 vs. 2.69±0.10, respectively, P<0.001, Average reduction: 36%, FIG. 9F). By days 14 and 28, there was no difference in the mean gene expression of TIMP-1 and TIMP-2 between both groups (P=NS). The average gene expression of TIMP-1 and TIMP-2 at the outflow vein correlated strongly with the length of time of fistula or graft placement for both LV-shRNA-VEGF-A and control shRNA groups (Spearman rank: r=1).

Adventitial transfection of LV-shRNA-VEGF-A to the Outflow Vein Reduces Gene Expression of ADAMTS-1 at Days 7 and 14

The following was performed to assess gene expression for ADAMTS-1 by RT-PCR analysis on specimens removed from the outflow vein that had been previously transfected with either LV-shRNA-VEGF-A or control shRNA at day 7, 14, and 28. By day 7, the mean gene expression of ADAMTS-1 (FIG. 9G) at the LV-shRNA-VEGF-A transfected vessels was significantly lower than the control vessels (0.89±0.19 vs. 2.0±0.2, respectively, P<0.05, Average reduction: 55%), which remained significantly lower at day 14 in the LV-shRNA-VEGF-A transfected vessels when compared to control vessels (0.29±0.03 vs. 0.42±0.06, respectively, P<0.05, Average reduction: 31%). The decrease in the gene expression at day 14 was statistically significant, however, the biologic consequences of it remain unknown. By day 28, the difference in ADAMTS-1 expression between the two groups was the same (P=NS). The average gene expression of ADAMTS-1 at the outflow vein correlated strongly with the length of time of fistula or graft placement for the LV-shRNA-VEGF-A and control shRNA groups (Spearman rank: r=1).

### Adventitial Transfection of LV-shRNA-VEGF-A to the Outflow Vein Decreases α-SMA Expression

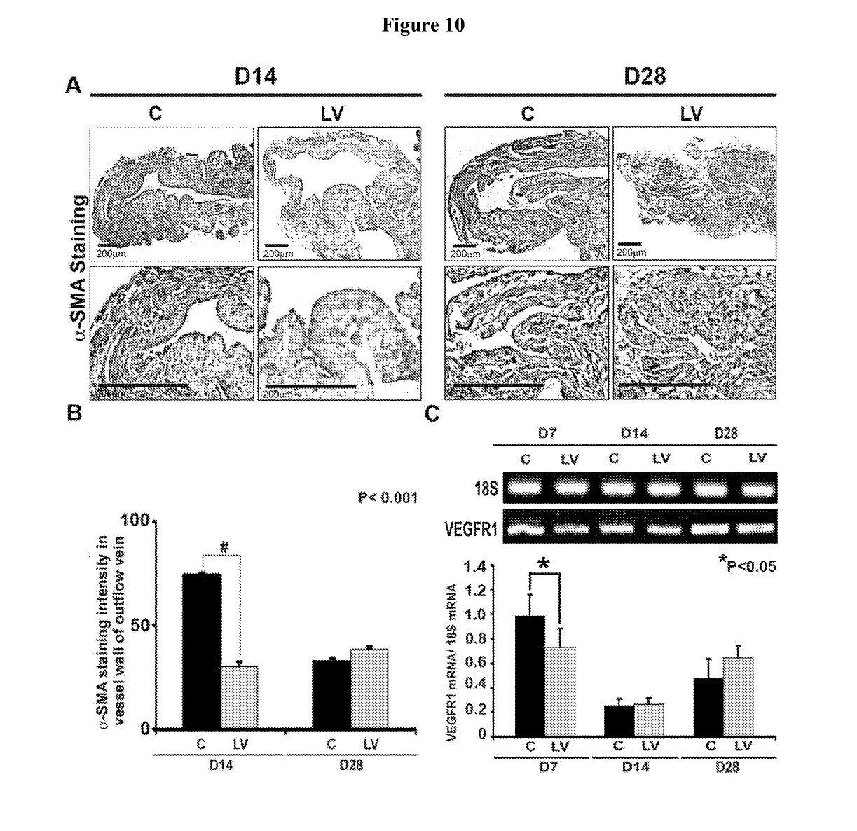

The majority of the cells comprising VNH have a α-SMA positive phenotype. The following was performed to determine if the decrease in the cell density was due to a decrease in α-SMA positive cells (FIG. 10A). By day 14, the average intensity of cells staining positive for α-SMA (pink) at the outflow vein of LV-shRNA-VEGF-A transfected vessels was significantly lower than the control group (30±2.3 vs. 74±1.2, respectively, P<0.001, Average reduction: 59%, FIG. 10B). By day 28, there were no differences in the α-SMA staining between the two groups. The average α-SMA expression at the outflow vein correlated strongly with the length of time of fistula or graft placement for the LV-shRNA-VEGF-A and control shRNA groups (Spearman rank: r=1).

Smooth muscle cells express VEGFR-1. The mean gene expression for VEGFFR-1 by day 7 at the LV-shRNA-VEGF-A transfected vessels was significantly lower than the control group (0.74±0.007 vs. 1.03±0.06, respectively, P<0.05, Average reduction: 29%, FIG. 10C). By days 14 and 28, there was no difference between the two groups (P=NS). The average gene expression of VEGFR-1 at the outflow vein correlated strongly with the length of time of fistula or graft placement for the LV-shRNA-VEGF-A and control shRNA groups (Spearman rank: r=1).

VEGF-A Silencing In Vitro is Associated with Decreased mRNA Levels of HIF-1a

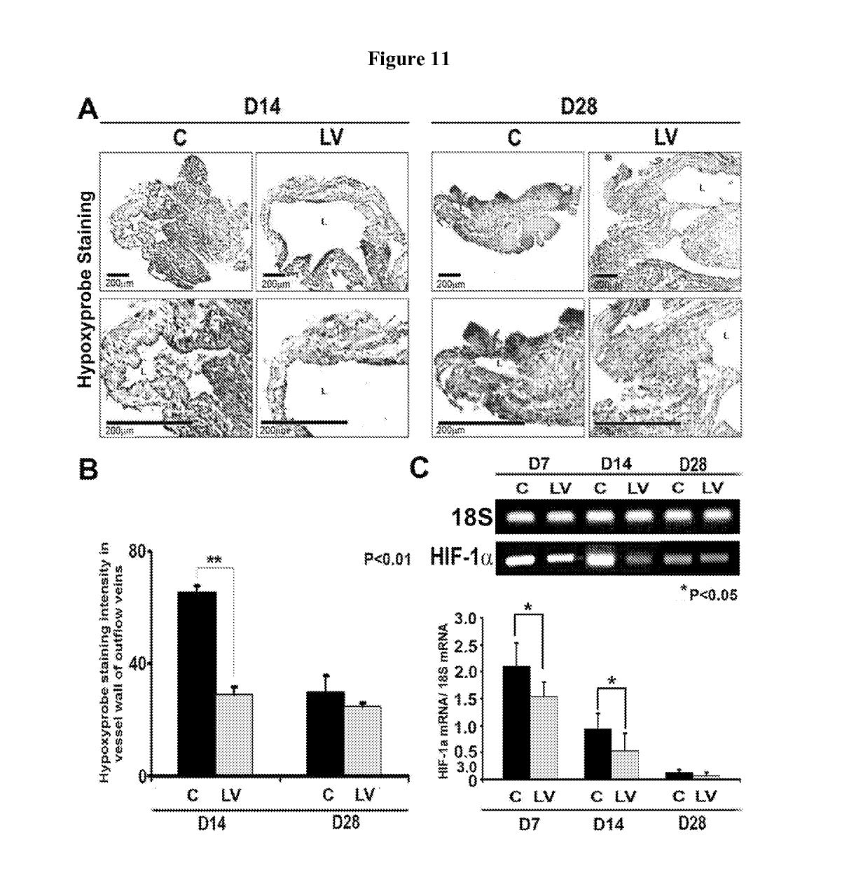

The hypoxic regions in the outflow vein were determined by staining for hypoxyprobe (FIG. 11A). Semiquantitative analysis for cells staining positive for hypoxyprobe (brown) was performed on sections from the outflow veins, which demonstrated that there was significant reduction in average intensity of hypoxyprobe staining at day 14 in the LV-shRNA-VEGF-A transfected vessels when compared to controls (30.7±2.4 vs. 65±2.4, respectively, P<0.01, Average reduction: 53%, FIG. 11B). By day 28, the average intensity of hypoxyprobe staining remained lower in the LV-shRNA-VEGF-A transfected vessels when compared to controls, however, it was not significant. The average intensity of hypoxyprobe staining correlated strongly with the length of fistula or graft placement (Spearman rank: r=1) for LV-shRNA-VEGF-A or control shRNA.

Because increased expression of HIF-1α has been observed in animal models of hemodialysis graft failure and in clinical specimens from patients with chronic graft failure, the expression levels for HIF-1α in outflow vein sections transfected with either LV-shRNA-VEGF-A or control shRNA were determined. By day 7, the mean gene expression of HIF-1α (FIG. 11C) at the LV-shRNA-VEGF-A transfected vessels was significantly lower than the control vessels (1.48±0.12 vs. 2.14±0.21, respectively, P<0.05, Average reduction: 33%) and remained significantly lower at day 14 in the LV-shRNA-VEGF-A transfected vessels when compared to controls (0.64±0.1 vs. 1.04±0.06, respectively, P<0.01, Average reduction: 39%). The average gene expression of HIF-1α correlated strongly with the length of time for the fistula or graft placement (Spearman rank: r=1) with either LV-shRNA-VEGF-A or control shRNA.

### VEGF-A Silencing in Hypoxic Fibroblasts Reduces α-SMA Production at 24 and 72 Hours

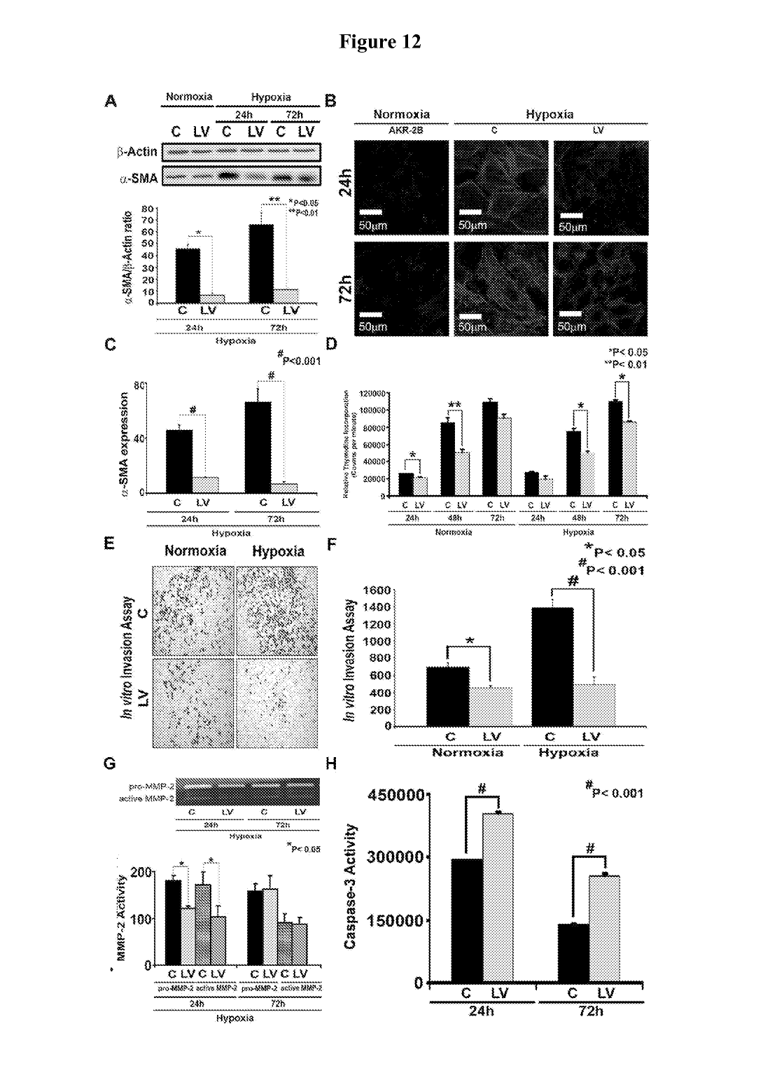

The following was performed to determine whether reducing VEGF-A gene expression in fibroblasts and then subjecting them to hypoxia would cause a decrease in α-SMA production when compared to controls with normal VEGF-A gene expression and normoxia. Murine AKR-2B cells transfected with either LV-shRNA-VEGF-A (LV) or control shRNA-VEGF-A (C) were subjected to 24 hours or 72 hours of hypoxia. Expression of α-SMA in the cell lysate was determined using Western blot analysis. The results indicate a significant reduction in α-SMA production at 24 hours (17.7±1.5 vs. 45.9±4.6, LV-shRNA-VEGF-A vs. control shRNA, respectively, P<0.001, Average reduction was 61%) and 72 hours (17±3 vs. 66.3±9.2, LV-shRNA-VEGF-A vs. control shRNA, respectively, P<0.001, Average reduction: 74%) of hypoxia when compared to control (FIG. 12A). The average expression of α-SMA production correlated strongly with the length of time for hypoxia in cells transfected with LV-shRNA-VEGF-A or control shRNA (Spearman rank: r=1).

Confocal microscopy for α-SMA staining was performed on AKR-2B cells transfected with either LV-shRNA-VEGF-A or control shRNA that had been subjected to 24 or 72 hours of hypoxia and demonstrated similar results (FIG. 12B). Semiquantitative analysis for cells staining positive for α-SMA (red) demonstrated a significant decrease in the intensity of the α-SMA staining in the LV-shRNA-VEGF-A transfected cells when compared to controls at 24 (132±5.8 vs. 179±3.4, respectively, P<0.001, average reduction: 27%) and 72 (18.7±9.42 vs. 100±9.5, respectively, P<0.001, average reduction: 71%) hours (FIG. 12C). The average intensity of α-SMA staining correlated strongly for the length of time for hypoxia (Spearman rank: r=1) in cells transfected with either LV-shRNA-VEGF-A or control shRNA.

### VEGF-A Silencing in Hypoxic Fibroblasts Reduces Proliferation and Invasion

The following was performed to determine if reducing VEGF-A gene expression in fibroblasts and subsequently subjecting them to hypoxia decreases the proliferative potential of fibroblasts when compared to controls. Murine AKR-2B cells transfected with either LV-shRNA-VEGF-A or control shRNA were subjected to normoxia and hypoxia, and a proliferation assay was performed which demonstrated a significant reduction in LV-shRNA-VEGF-A transfected cells when compared to control for normoxia at 24 and 48 hours with significant reduction in hypoxic cells at 48 and 72 hours (FIG. 12D).

The following was performed to determine if the invasive capacity of these cells was reduced under the same conditions. Murine AKR-2B cells transfected with either LV-shRNA-VEGF-A or control shRNA were subjected to normoxia and hypoxia, and an invasion assay was performed demonstrating a significant reduction in invasive capabilities of LV-shRNA-VEGF-A transfected cells when compared to control (Normoxia: 600±10 vs. 400±10, control shRNA vs. LV-shRNA-VEGF-A, respectively, P<0.05, Average reduction: 33%; Hypoxia: 1400±10 vs. 400±10, control shRNA vs. LV-shRNA-VEGF-A, respectively, P<0.001, Average increase: 350%, FIGS. 12E and 12F). The average expression of invasion correlated strongly with normoxia and hypoxia in cells transfected with LV-shRNA-VEGF-A or control shRNA (Spearman rank: r=1).

### VEGF-A Silencing in Hypoxic Fibroblasts Decrease MMP-2 Activity and Increases Caspase 3

Because there was a decrease in proliferation and invasion in cells transfected with LV-shRNA-VEGF-A, the following was performed to determine if there was a decrease in MMP-2 expression using zymography. A significant decrease in the pro MMP-2 (122±5.4 vs. 180±11, LV-shRNA-VEGF-A vs. control shRNA, respectively, P<0.01, Average reduction: 33%) and active MMP-2 activity (103±24 vs. 171±28, LV-shRNA-VEGF-A vs. control shRNA, respectively, P<0.05, Average reduction: 33%) at 24 hours was observed, and by 72 hours, the pro and active MMP-2 activity had become the same in both groups (P=NS, FIG. 12G). The average pro and active MMP-2 activity correlated strongly with the length of time for hypoxia (Spearman rank: r=1) in cells transfected with LV-shRNA-VEGF-A or control shRNA.

Since VEGF-A is involved in cellular homesotasis, the following was performed to determine if reducing VEGF-A expression would result in an increase in caspase 3 activity (FIG. 12H). A significant increase in caspase 3 activity in LV-shRNA-VEGF-A transfected cells when compared to controls at 24 (405725±1013 vs. 292723±558, respectively, P<0.001, Average increase: 160%) and 72 (254277±5870 vs. 137980±2810, respectively, P<0.001, Average increase: 184%) hours of hypoxia was observed. The average caspase 3 activity correlated strongly for length of time for hypoxia (Spearman rank: r=1) in cells transfected either LV-shRNA-VEGF-A or control shRNA.

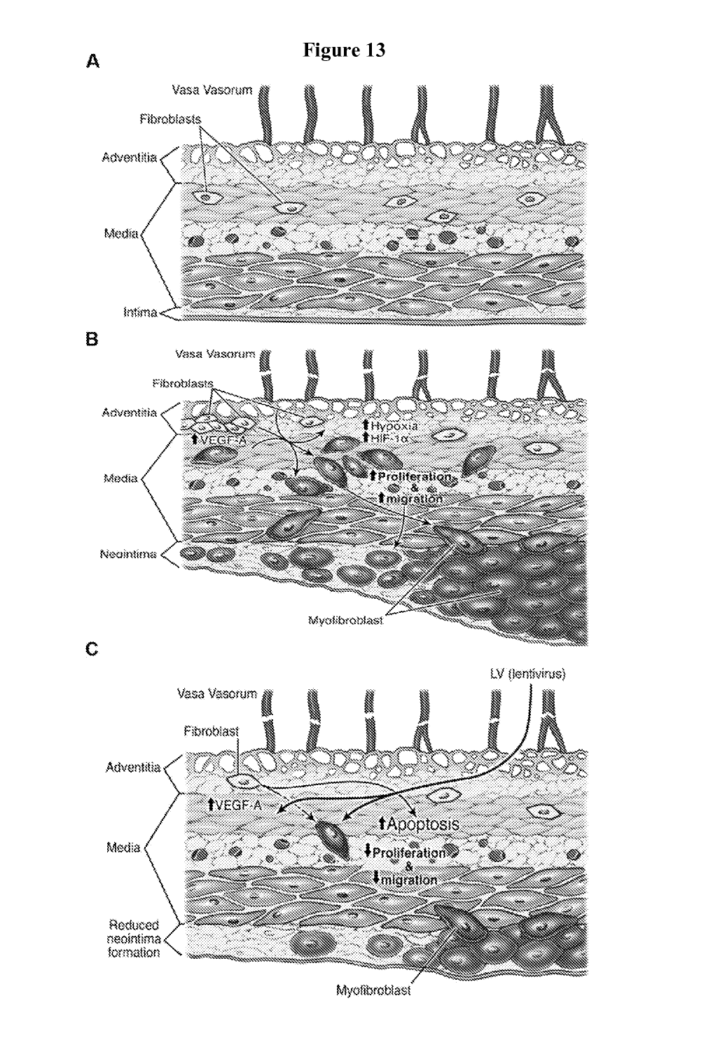

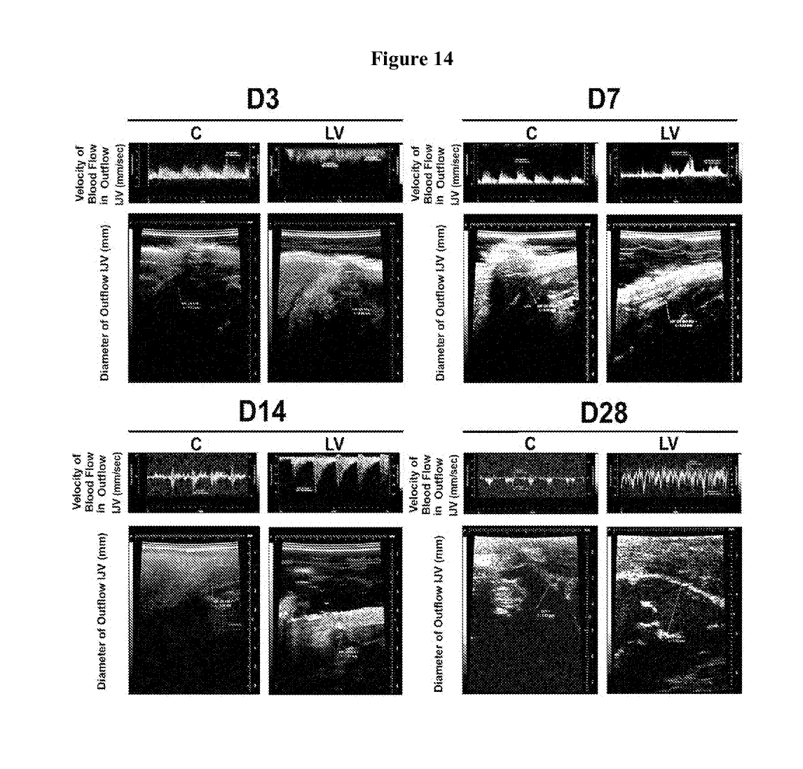

The results provided herein indicate that VNH formation occurs in part because of local vessel hypoxia caused by surgical trauma to the vasa vasorum supplying the outflow vein at the time of AVF placement. This hypoxia in turn can lead to an increase in gene expression of VEGF-A, MMP-2, MMP-9, and ADAMTS-1, and the resulting activation of adventitial fibroblast, which undergo conversion to myofibroblasts with increased proliferative and migratory capacity, thereby resulting in VNH formation (FIG. 13B).

The results provided herein demonstrate that selective targeting of the adventitia of the outflow vein using an anti-VEGF-A therapy (e.g., LV-shRNA-VEGF-A) at the time of fistula or graft creation can prevent venous stenosis formation. The result of decreasing mRNA of VEGF-A had two functional consequences. First, there was an increase in apoptosis at the outflow vein. Second, there was a decrease in cellular proliferation. An increase in apoptosis was accompanied by an increase in caspase 3 activity and TUNEL staining with a decrease in cellular density, in particular of cells staining positive for α-SMA with concomitant decrease in VEGFR-1 expression.

The decrease in cellular proliferation was reflected by a decrease in Ki-67 staining and a decrease in VEGF-A associated signaling moieties including MMP-2, MMP-9, TIMP-1, TIMP-2, and ADAMTS-1. It is hypothesized that the net effect of decreasing cellular proliferation and increasing cellular apoptosis results in a decrease in local vessel oxygen demand and subsequent decrease in mRNA levels for HIF-1α and hypoxyprobe staining. At early time points, a decrease in cells staining positive for α-SMA and HIF-1α in the LV-shRNA-VEGF-A transfected cells was observed when compared to controls.

The results provided herein also suggest a potential cellular mechanism for the in vivo observations (FIG. 13C) and indicate that adventitial delivery of LV-shRNA-VEGF-A decreases expression of several pro-migratory cytokines such as VEGFR-1, MMP-2, MMP-9, and ADAMTS-1. The net result of these interventions is an overall decrease in cell proliferation of α-SMA positive cells and an increased apoptosis with positive vascular remodeling. The clinical significance of these results is that it provides rationale for using anti-VEGF-A therapies such as tyrosine kinase inhibitors at the time of fistula or graft creation to reduce VNH formation.

### Example 2

### Simvastatin Reduces Venous Stenosis Formation in a Hemodialysis Vascular Access Model

### Experimental Animals

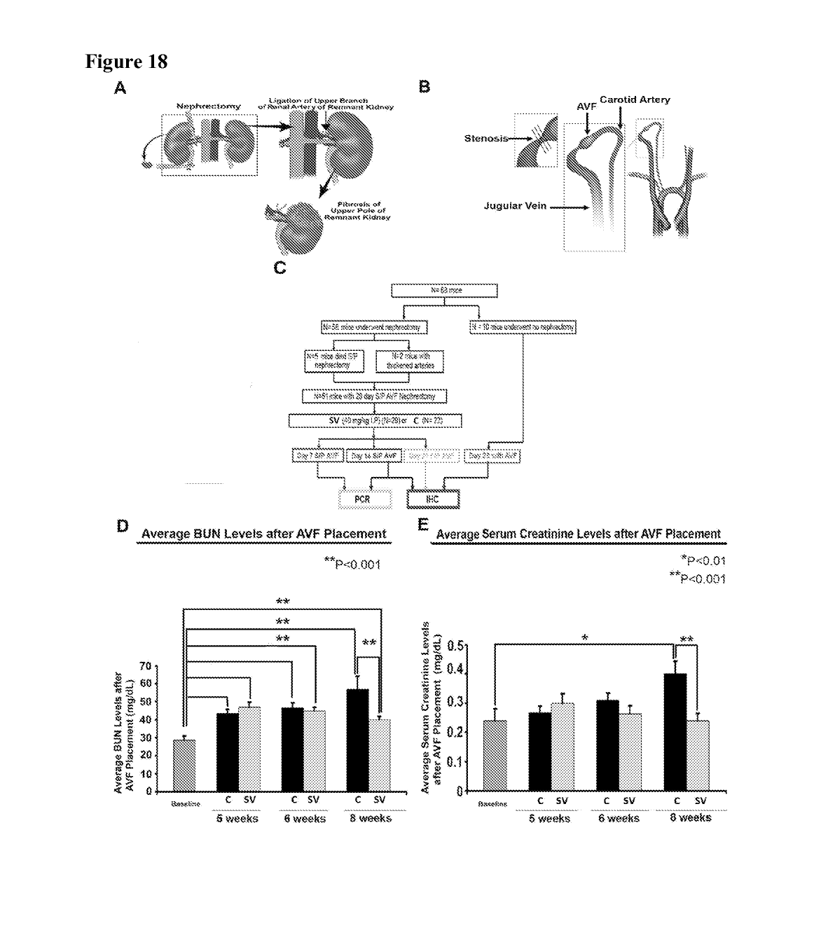

Animals were housed at 22° C. temperature, 41% relative humidity, and 12-/12-hour light/dark cycles. Animals were allowed access to water and food ad libitum. Anesthesia was achieved with intraperitoneal injection of a mixture of ketamine hydrochloride (0.20 mg/g) and xylazine (0.02 mg/g) and maintained with intraperitoneal pentobarbital (20-40 mg/kg). Sixty-eight male C57BL/6 mice (Jackson Laboratories, Bar Harbor, Me.) weighing 25-30 grams were used for the present study (FIG. 18). Chronic kidney disease was created by surgical removal of the right kidney accompanied by ligation of the arterial blood supply to the upper pole of the left kidney as described elsewhere (Misra et al., J. Vasc. Interv. Radiol., 21:1255-1261 (2010); see, also, FIG. 18A). Three weeks after nephrectomy, the animals were started on simvastatin (40 mg/kg administered i.p. three times per week) or PBS (equal amount of volume used for simvastatin i.p. controls). Simvastatin was prepared as described elsewhere (Wilson et al., Arterioscler Thromb. Vasc. Biol., 21:122-128 (2001)). A week later, an AVF was created by connecting the right carotid artery to the ipsilateral jugular vein (FIG. 18B; see, also, Misra et al., J. Vasc. Interv. Radiol., 21:1255-1261 (2010) and Yang et al., J. Vasc. Interv. Radiol., 20:946-950 (2009)). Animals were sacrificed at day 7, day 14, and day 28 following AVF placement for real time polymerase chain reaction (RT-PCR) and histomorphometric analyses (FIG. 18C). Serum BUN and creatinine were measured by removing blood from the tail vein at baseline (before nephrectomy), at AVF creation, and at the time of sacrifice. A separate group of experiments were conducted in mice that did not undergo nephrectomy. These animals were started on simvastatin (40 mg/kg i.p.) or PBS (equal amount of volume i.p.) every other day one week before AVF placement and then sacrificed four weeks after fistula placement for histomorphometric analysis.

### Tissue Harvesting

At euthanasia, all mice were anesthetized, and the arterio-venous fistula was carefully dissected from of the surrounding tissue. Animals were euthanized by use of CO₂ asphyxiation, and the outflow veins harvested for RT-PCR or histomorphometric analyses as described elsewhere (Misra et al., Kidney Int., 68:2890-2900 (2005); Misra et al., J. Vasc. Interv. Radiol., 21:1255-1261 (2010); and Misra et al., Am. J. Physiol. Heart Circ. Physiol., 294:H2219-2230 (2008)).

### RNA Isolation

The tissue was stored in RNA stabilizing reagent (Qiagen, Gaithersburg, Md.) as per the manufacturer's guidelines. To isolate the RNA, the specimens were homogenized, and total RNA was isolated using RNeasy mini kit (Qiagen) (Misra et al., J. Vasc. Interv. Radiol., 21:1255-1261 (2010); and Yang et al., J. Vasc. Interv. Radiol., 20:946-950 (2009)).

### Real Time Polymerase Chain Reaction (RT-PCR) Analysis

Expression for the gene of interest was determined using RT-PCR analysis as described elsewhere (Misra et al., J. Vasc. Interv. Radiol., 21:1255-1261 (2010)). Commercial PCR primers for the gene of interest were purchased from SA Biosciences (Frederick, Md.).

### Immunohistochemistry for Ki-67 or α-SMA

The outflow vein with the cuff anastomosis was harvested as shown in FIG. 18B and then embedded. Multiple serial four-micrometer sections were stained and analyzed. Cellular proliferation was determined by staining for Ki-67 on sections removed from the outflow vein for simvastatin treated vessels or control groups. Smooth muscle density (α-SMA) or proliferation index (Ki-67) was determined by staining on sections removed from the outflow vein by performing quantification at the different time points. Immunohistochemistry for Ki-67 and α-SMA was performed on paraffin-embedded sections from the outflow vein after transfection with either simvastatin or controls using the Vectastain Elite ABC system (Vector Laboratories, Burlingame, Calif., USA). The following antibodies were used: mouse monoclonal antibody Ki-67 (DAKO, Carpentaria, Calif.; 1:400) or rabbit polyclonal antibody to mouse for α-SMA (Abcam, Cambridge, Mass.; 1:400).

### Hypoxyprobe Staining at Day 14 and 28

Hypoxic changes were assessed in the outflow vein of simvastatin treated vessels or control groups using Hypoxyprobe™-1 (EMD Millipore, Billerica, Mass.), a substituted derivative of pimonidazole hydrochloride. Hypoxyprobe™-1 upon activation forms stable covalent adducts with thiol groups of polypeptides and amino acids of hypoxic tissue. Mice were injected with 60 mg/kg Hypoxyprobe™-1 i.p. Thirty minutes following injection, mice were sacrificed, and outflow veins were dissected and fixed as specified for histological analysis. Four-micrometer thick paraffin embedded sections were stained with the anti-hypoxyprobe-1 Ab as per the manufacturer's directions.

### TUNEL Staining at Day 14 and 28

TUNEL staining was performed on paraffin-embedded sections from the outflow vein after treatment with either simvastatin or controls as specified by the manufacturer (DeadEnd Colorimetric tunnel assay system, G7360, Promega, Madison, Wis.).

### Picrosirius Red Staining at Day 14 and 28

The paraffin embedded sections were de-waxed and hydrated before being stained with picrosirius red for one hour to achieve a near-equilibrium staining. The sections were then washed twice with acidified distilled water before being subjected to dehydration process in sequential grades of alcohol before being mounted in a resinous medium.

### Hypoxia Chamber

One hundred thousand NIH 3T3 cells were treated with simvastatin (1 μM, 5 μM, or 10 μM) or controls and made hypoxic for 24 hours as described elsewhere (Misra et al., J. Vasc. Interv. Radiol., 21:896-902 (2010)).

### Western Blot of α-SMA

The differentiation of fibroblasts to myofibroblasts was assessed by performing Western blot analysis for α-SMA. The cultured cells were processed for Western analysis using rabbit polyclonal antibody as described elsewhere (Misra et al., J. Vasc. Interv. Radiol., 19:252-259 (2008)).

### Proliferation Assay

One hundred thousand NIH 3T3 cells were treated with simvastatin or controls and made hypoxic for 24 hours. Next, they were seeded in a 6-well plate and cultured for 24 hours in DMEM medium. After 20 hours, 1 mCi of (³H) thymidine was added to each well. Four hours later, the cells were washed with chilled PBS, fixed with 100% cold methanol, and collected for measurement of trichloroacetic acid-precipitable for radioactivity. Experiments were repeated three times for each time point.

### Cell Migration Assay

NIH 3T3 cells were synchronized for 24 hours in serum free media. Next, one hundred thousand NIH 3T3 cells were treated with simvastatin or controls and seeded in 8-micron trans-wells that were pre-coated with low growth factor matrigel in a serum free media. The complete media was supplemented under the trans-well and incubated for 6 hours at 37° C. After 6 hours, trans-wells were washed with PBS and fixed with paraformaldehye (4% v/v). Finally, trans-wells were stained with bromophenol (0.1%) solution. The cells from upper side were removed with cotton tip applicators. The cells at bottom side were counted for analysis.

### Caspase 3

Apoptosis was assessed using an ELISA assay for caspase 3. Cellular protein was extracted from one hundred thousand cultured cells as described elsewhere (Misra et al., J. Vasc. Interv. Radiol., 21:896-902 (2010)). The enzymatic activity of caspase 3 was accessed by Caspase Glo assay (G811C, Promega, Madison, Wis.).

### Morphometry and Image Analysis

Morphometric analysis was performed as described elsewhere (Misra et al., Kidney Int., 68:2890-2900 (2005); and Misra et al., Am. J. Physiol. Heart Circ. Physiol., 294:H2219-2230 (2008)). Briefly, the outflow vein was sectioned into multiple contiguous 4-μm paraffin embedded sections. Typically, 2 to 3 sections per animal for each group (simvastatin and controls) and time point (day 14 and day 28) were photographed and then quantified using KS 400 (Carl Zeiss, Inc., Thornwood, N.Y.) semiquantitative program.

### Statistical Methods

Data were expressed as mean±SEM. Multiple comparisons were performed with two-way ANOVA followed by Student t-test with post hoc Bonferroni's correction. Because of the Bonferroni correction, significant difference from control value was indicated by *P<0.01, **P<0.001, or ^{#}P<0.0001. SAS version 9 (SAS Institute Inc., Cary, N.C.) was used for statistical analyses.

### Results

### Surgical Outcomes

Sixty-eight male C57BL/6 mice weighing 25-30 were used. Of the sixty-eight mice, fifty-eight underwent right nephrectomy and left upper pole occlusion surgery (FIG. 18A). Five mice died after nephrectomy, and two had significant arterial thickening and inflammation such that a new AVF could not be placed. Fifty-one mice underwent placement of an AVF to connect the right carotid artery to the ipsilateral jugular vein (FIG. 18B). Next, either 40 mg/g of simvastatin (SV, n=29) or PBS only (control, C, n=22) was given i.p. every other day starting one week before fistula placement until sacrifice. Animals were sacrificed for gene expression or histomorphometric analyses at day 7, 14, and 28 after AVF placement (FIG. 18C). In order to determine the effect of simvastatin alone on venous neointimal hyperplasia, a group of ten animals that did not undergo nephrectomy received either simvastatin or control one week prior to AVF placement and were sacrificed 28 days after fistula placement for histomorphometric analyses only.

### Serum BUN and Creatinine After Nephrectomy

The kidney function after nephrectomy was determined by measuring the serum BUN and creatinine. The average BUN post-nephrectomy was significantly increased for the simvastatin and control group at all time points (P<0.001) when compared to baseline (FIG. 18E). At 8-weeks post-nephrectomy, the average BUN had decreased in the simvastatin group and was similar to that measured in the control group. The average serum creatinine post-nephrectomy was also significantly increased in the control group at 8-weeks (P<0.01). 8-weeks following nephrectomy, the average serum creatinine had decreased in simvastatin treated animals and was similar to that found in controls (P<0.0001).

Simvastatin Treated Vessels have a Significant Reduction in Average Gene Expression of VEGF-A at the Outflow Vein at Day 7 and 14

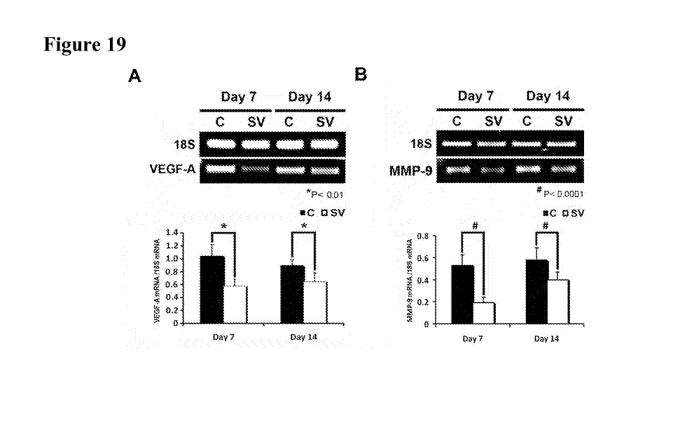

VEGF-A expression is increased in specimens removed from either patients with failed hemodialysis vascular accesses (AV Fistulas or AV Grafts) and in experimental animal models (Roy-Chaudhury et al., Kidney Int., 59:2325-2334 (2001); Misra et al., Kidney Int., 68:2890-2900 (2005); Misra et al., J. Vasc. Interv. Radiol., 21:1255-1261 (2010); and Misra et al., Am. J. Physiol. Heart Circ. Physiol., 294:H2219-2230 (2008)). By day 7, the mean gene expression of VEGF-A at the simvastatin treated vessels was significantly lower than the control vessels (0.58±0.06 vs. 1.04±0.1, respectively, P<0.01, Average reduction: 44%, FIG. 19A). At day 14, the mean gene expression of VEGF-A at the simvastatin treated vessels was significantly lower than the control vessels (0.51±0.1 vs. 1.03±0.07, respectively, P<0.01, Average reduction: 49%). Taken together, these results indicate that the average gene expression of VEGF-A is reduced at the outflow vein in simvastatin treated vessels when compared to control vessels.

Simvastatin Treated Vessels have a Significant Reduction in Gene Expression of MMP-9 at the Outflow Vein at Day 7 and 14

The average gene expression of MMP-2 was not significantly different in the simvastatin treated vessels when compared to controls. The average gene expression of MMP-9 was significantly lower in the simvastatin treated vessels when compared to controls by day 7 (0.19±0.03 vs. 0.62±0.02, respectively, P<0.0001, Average reduction: 69%, FIG. 19B) and by day 14 (0.37±0.02 vs. 0.63±0.02, respectively, P<0.0001, Average reduction: 41%). Overall, these results indicate that that simvastatin treated vessels have a significant reduction in MMP-9 when compared to control vessels.

Kidneys Treated in Simvastatin Treated Animals have Decreased Gene Expression of VEGF-A, MMP-2, and MMP-9 at 4-Weeks

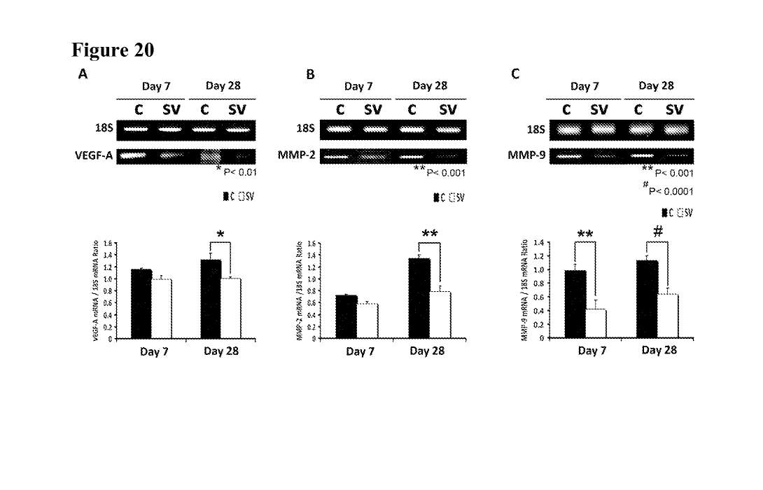

The following was performed to determine if the improvement in kidney function was due to a decrease in genes implicated in causing chronic kidney disease such as VEGF-A (FIG. 20A), MMP-2 (FIG. 20B), and MMP-9 (FIG. 20C). The average gene expression of VEGF-A was significantly reduced at day 28 (1±0.02 vs. 1.32±0.11, respectively, P<0.01, Average reduction: 24%) in the simvastatin treated kidneys when compared to controls. Next, the expression of MMP-2 was examined. By day 28, it became significantly lower in the simvastatin treated kidneys when compared to controls (0.78±0.09 vs. 1.34±0.06, respectively, P<0.001, Average reduction: 42%). Finally, the expression of MMP-9 was examined. The average gene expression of MMP-9 was significantly lower in the simvastatin treated kidneys when compared to controls by day 7 (0.42±0.13 vs. 0.98±0.09, respectively, P<0.01, Average reduction: 57%, FIG. 20C) and by day 28 (0.64±0.09 vs. 1.14±0.06, respectively, P<0.0001, Average reduction: 44%). Taken together, these results indicate that the average gene expression of VEGF-A, MMP-2, and MMP-9 are significantly reduced in the simvastatin treated kidneys when compared to controls.

Simvastatin Treated Vessels have Positive Vascular Remodeling at Days 14 and 28

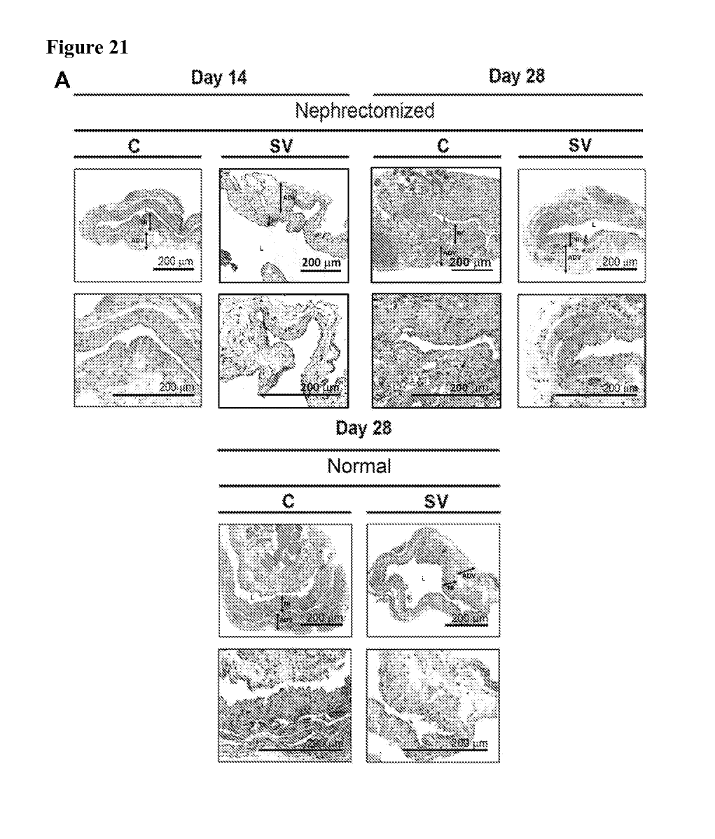

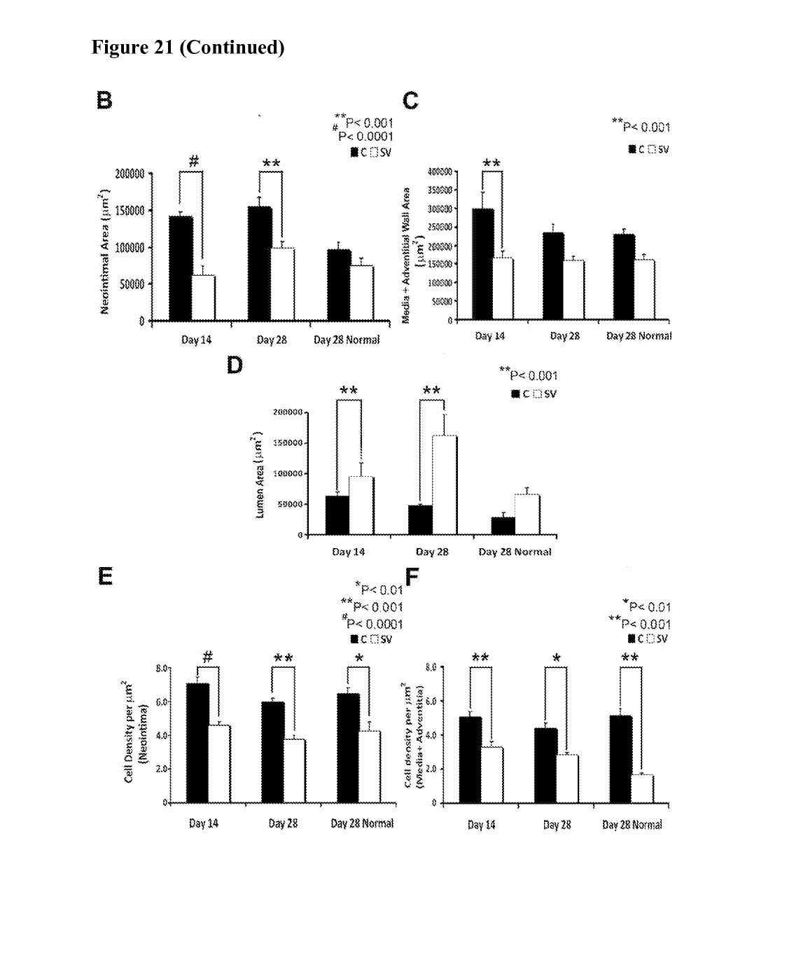

It was hypothesized that simvastatin treated vessels would have reduced venous neointimal hyperplasia when compared to control vessels (FIG. 21A). On hematoxylin and eosin stained sections, it was possible to differentiate between the neointima (NI) and media/adventitia (ADV). Semiquantitative histomorphometric analysis was performed on sections removed from the outflow veins of simvastatin treated vessels and control vessels at day 14 and 28 after nephrectomy, and day 28 in normal (animals without nephrectomy) for the following vascular remodeling measures including the area of the neointima (FIG. 21B), media/adventitia (FIG. 21C), and lumen vessel (FIG. 21D). There was a significant reduction in the average area of the neointima of the simvastatin treated vessels when compared to the controls by day 14 (61864±12401 μm² vs. 141924±5613, respectively, average reduction: 56%, P<0.0001), and by day 28 (85574±1652 vs. 154691±12406, respectively, average reduction: 45%, P<0.001). No difference was observed between the two groups at day 28 normal.

Next, the average area of the media/adventitia (FIG. 21C) was determined in simvastatin treated vessels and compared to controls. The average area of the media/adventitia was significantly lower in the simvastatin treated vessels when compared to the control group by day 14 (147603±4443 μm² vs. 29903 8±44318, respectively, P=0.0028, Average reduction: 43%). There was no statistical significant difference at day 28 (Average reduction: 32%, P=0.03) and day 28 normal (Average reduction: 29%, P=0.03).

Since the simvastatin treated vessels had reduced average wall area when compared to controls, the following was performed to determine if the simvastatin treated vessels had a larger average lumen vessel area. The average lumen vessel area was significantly higher in the simvastatin treated vessels when compared to controls by day 14 (95051±21583 vs. 63315±6654, respectively, P<0.001, average increase: 150%) and by day 28 (162607±34685 vs. 47352±2293, respectively, average increase: 343%, P<0.001) (FIG. 21D). No difference between the two groups was observed for the day 28 normal group.

The following was performed to determine if the decrease in wall area with increase in the lumen vessel area in the simvastatin treated group was due to a decrease in cell density at the outflow vein. Quantitative analysis was performed for average cell density in the neointima and media/adventitia in the simvastatin treated vessels at days 14 and 28 after AVF placement and day 28 without nephrectomy. The average cell density of the neointima in the simvastatin treated vessels was significantly lower than the control vessels by day 14 (4.6±0.2 vs. 7.1±0.4, respectively, P<0.0001, average reduction: 65%), by day 28 (4.2±0.2 vs. 6.0±0.19, respectively, P<0.001, average reduction: 70%), and by day 28 normal (2.63±0.11 vs. 6.7±0.28, respectively, P<0.01, average reduction: 61%) (FIGS. 21E and 21F).

The average cell density of the media/adventitia also was determined. The average cell density of the media/adventitia in the simvastatin treated vessels was significantly lower than the control specimens by day 14 (3.27±0.33 vs. 5.12±0.36, respectively, P<0.001, average reduction: 37%), by day 28 (2.85±0.10 vs. 4.41±0.27, respectively, P<0.01, average reduction: 35%), and by day 28 normal (1.67±0.1 vs. 5.77±0.03, respectively, P<0.001, average reduction: 71%).

Vessels in Simvastatin Treated Animals have Increased TUNEL Staining at Day 14 and 28

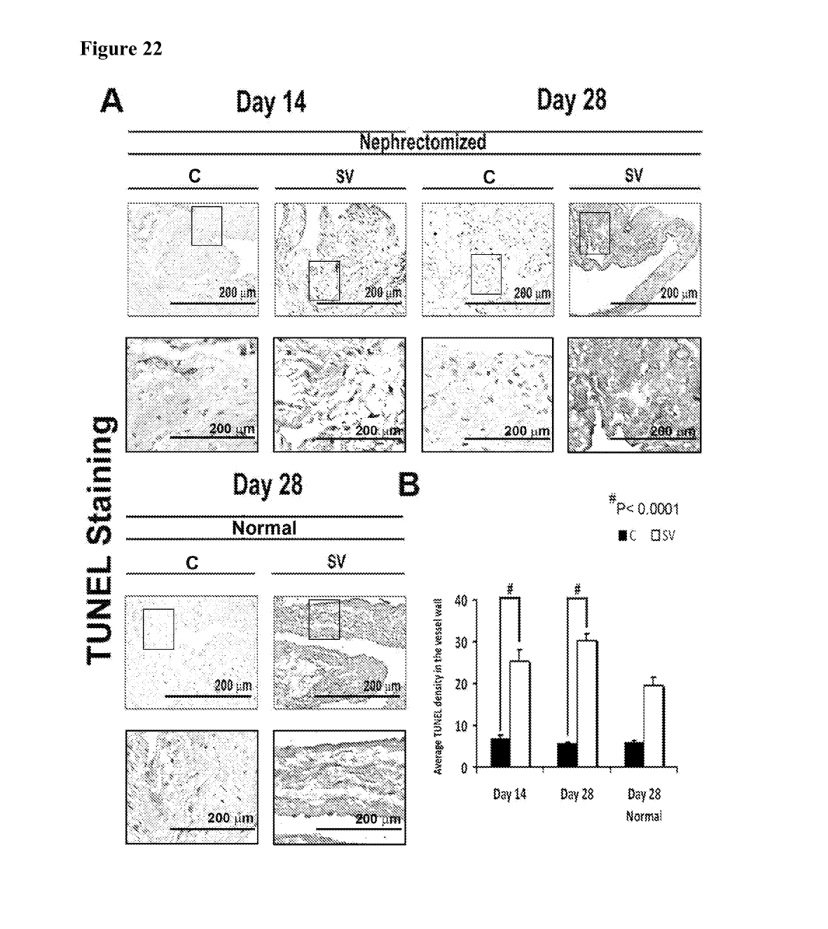

It was hypothesized that the decrease in cell density was due to an increase in apoptosis. Apoptosis was assessed by performing TUNEL staining in sections removed from the outflow vein at day 14 and 28 in the simvastatin treated vessels and control groups. The average density of cells staining positive for TUNEL (brown) at the outflow vein of the simvastatin group was significantly higher than the control group by day 14 (25.26±2.78 vs. 6.9±0.8, respectively, average increase: 366%, P<0.0001), by day 28 (30.17±1.8 vs. 5.38±0.24, respectively, P<0.0001, average increase: 561%), and was increased by 326% at day 28 normal (P=0.0136) (FIGS. 22A and 22B). Overall, these results indicate that simvastatin treated vessels have increased TUNEL activity implying cellular apoptosis when compared to controls.

Simvastatin Treated Vessels have Decreased Cellular Proliferation at the Outflow Vein at Day 14 and 28

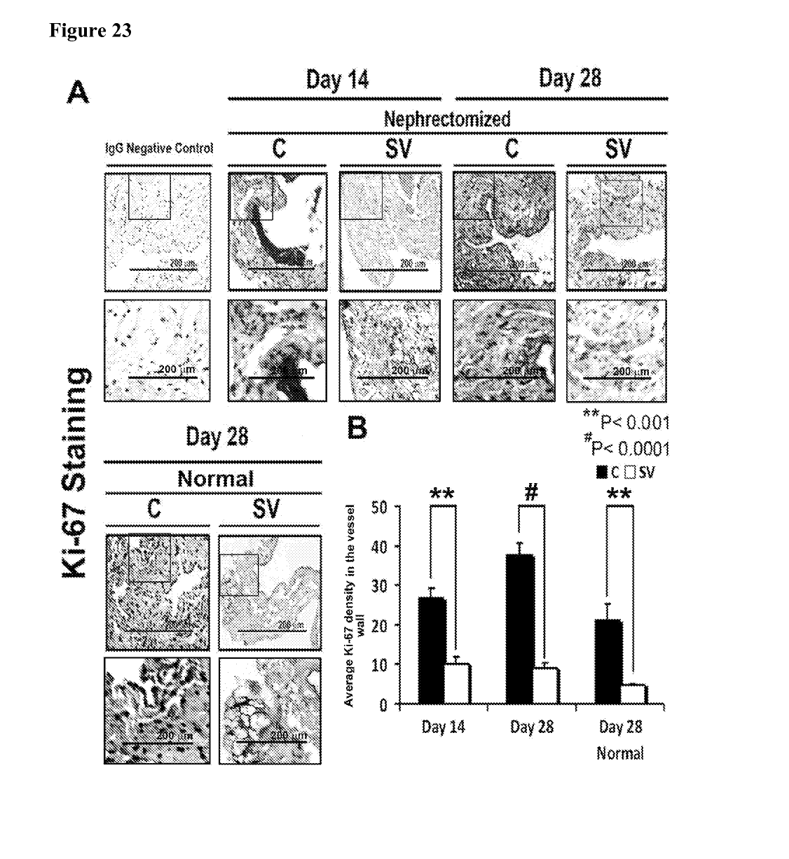

The following was performed to determine whether the decrease in cell density was due to a decrease in cell proliferation. Cells staining positive for Ki-67 had brown stained nuclei (FIG. 23A). Cellular proliferation was assessed using Ki-67. In the simvastatin treated vessels when compared to control vessels, the average Ki-67 density was significantly lower by day 14 (10.1±2.9 vs. 29.3±0.23, respectively, P<0.001, average reduction: 66%), by day 28 (9.1±1.3 vs. 37.8±2.2, respectively, P<0.0001, average reduction: 76%), and by day 28 normal (4.83±0.2 vs. 21.2±2.3, respectively, P<0.001, average reduction: 77%) (FIG. 23B).

Simvastatin Treated Vessels have Decreased α-SMA Expression by Day 14 and 28

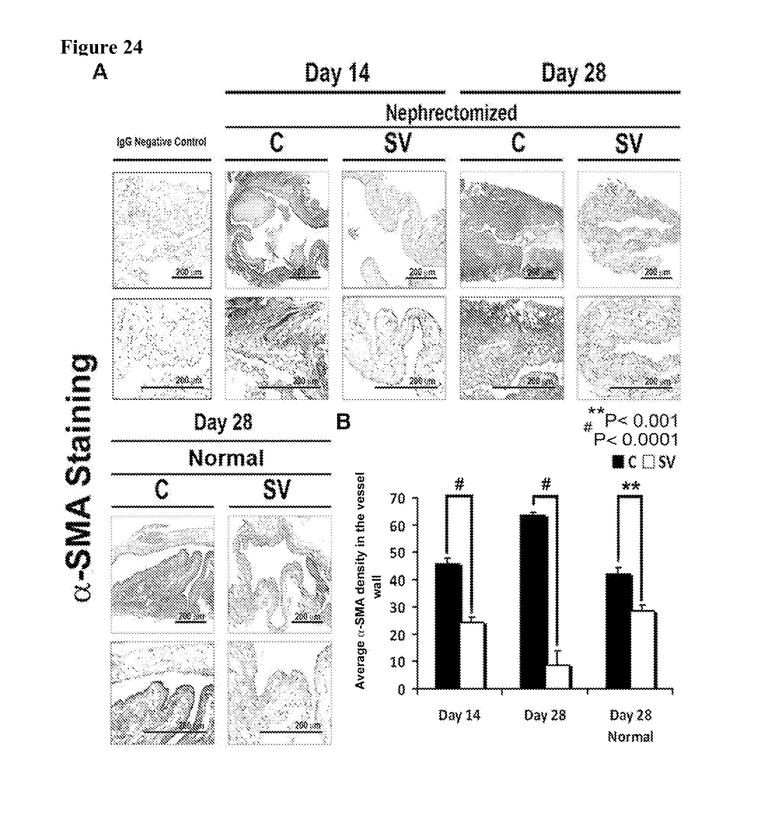

The majority of the cells which comprise the venous neointimal hyperplasia were α-SMA positive. Brown staining cells were positive for α-SMA, and it was determined if the decrease in the cell density was due to a decrease in α-SMA positive cells. The average α-SMA density at the outflow vein of simvastatin treated vessels was significantly lower than the control group by day 14 (24±1.2 vs. 45±3.5, respectively, P<0.0001 average reduction: 46%), day 28 (8.4±1.6 vs. 63.6±0.8, respectively, P<0.0001, average reduction: 87%), and day 28 normal (28.4±1.6 vs. 42±1.4, respectively, P<0.01, average reduction: 32%) (FIGS. 24A and 24B).

Simvastatin Treated Vessels have Reduced Gene Expression of CTGF at Day 14

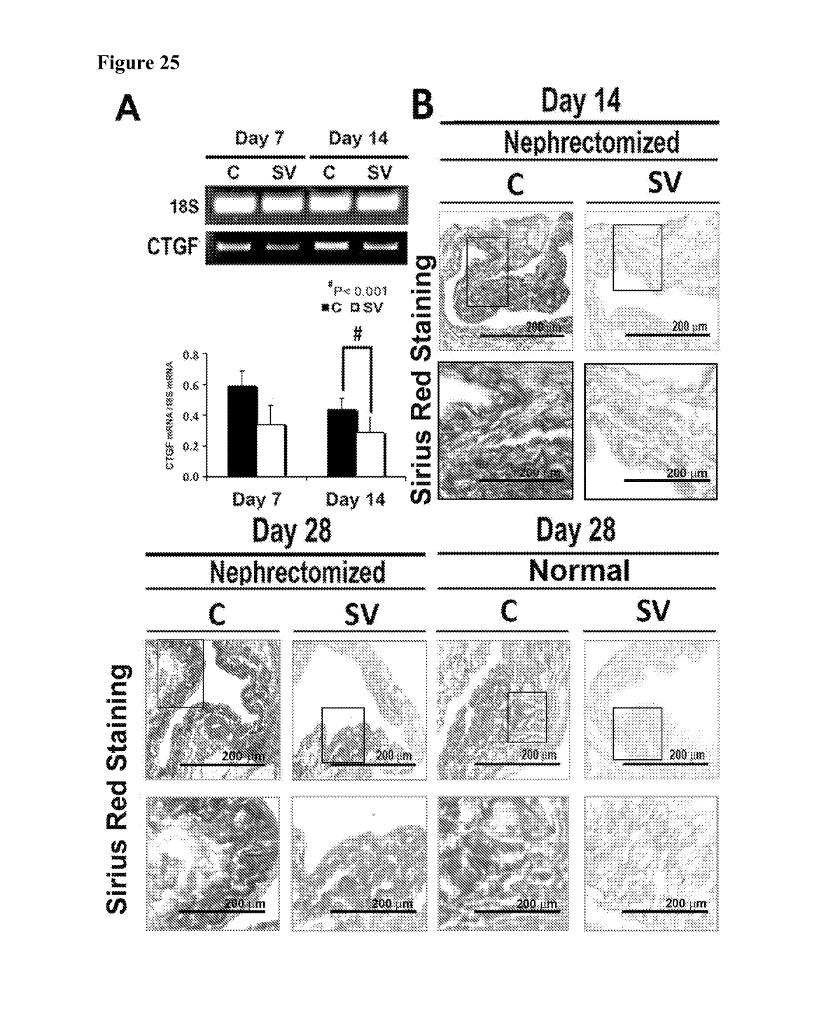

Several genes including connective tissue growth factor (CTGF) control the regulation of extracellular matrix. The gene expression of CTGF was assessed using RT-PCR analysis performed at different time points. The mean gene expression of CTGF at the simvastatin treated vessels was significantly lower than the control vessels by day 14 (0.29±0.05 vs. 0.52±0.04, respectively, P<0.001, average reduction: 45%). (FIG. 25A).

Simvastatin Treated Vessels have Reduced Sirrus Red Staining

The changes in extracellular matrix were assessed using Sirrus red staining, which allows for the evaluation of collagen 1 and 3. Sirrus red staining was performed on outflow veins sections removed from simvastatin treated and control vessels at day 14 and day 28. Qualitatively, this demonstrated a reduction in the intensity of Sirrus red staining in the simvastatin treated vessels when compared to control vessels at both day 14 and day 28 (FIG. 25B).

Simvastatin Treated Vessels have Decreased Hypoxyprobe Staining and Decreased mRNA Levels of HIF-1α

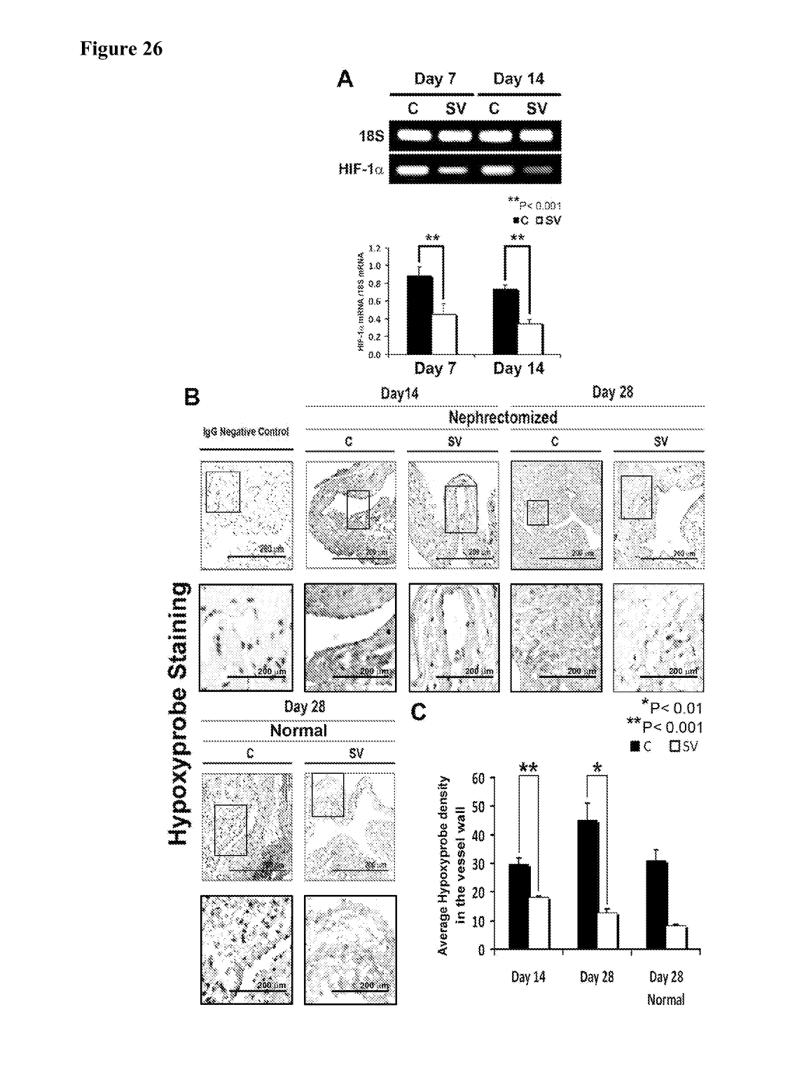

The mean gene expression of HIF-1α at the simvastatin treated vessels was significantly lower than the control vessels by day 7 (0.45±0.12 vs. 0.98±0.07, respectively, P<0.001, average reduction: 54%) and by day 14 (0.34±0.04 vs. 0.74±0.04, respectively, P<0.001, average reduction: 54%) (FIG. 26A).

Hypoxyprobe staining in the outflow vein treated with either simvastatin or controls was performed. Cells staining positive for hypoxyprobe are brown (FIG. 26B). There was significant reduction in the average density of hypoxyprobe staining in the simvastatin treated vessels when compared to controls by day 14 (18.33±2.06 vs. 28.66±1.11, respectively, P<0.001, average reduction: 53%), by day 28 (13.01±4.62 vs. 43.63±6.08, respectively, P<0.01, average reduction: 70%), and 72% reduced by day 28 normal (P=0.0103) (FIG. 26C). Overall these results indicate that there is decreased expression of both HIF-1α and hypoxyprobe simvastatin treated vessels when compared to controls.

### Simvastatin Treatment in Hypoxic Fibroblasts Reduces α-SMA Production at 24 Hours

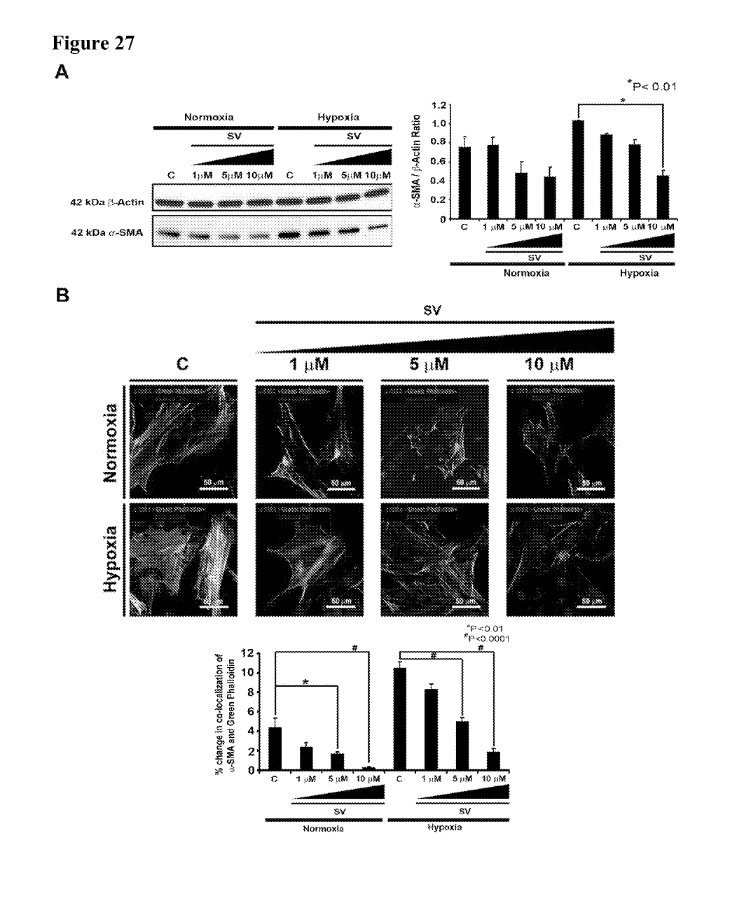

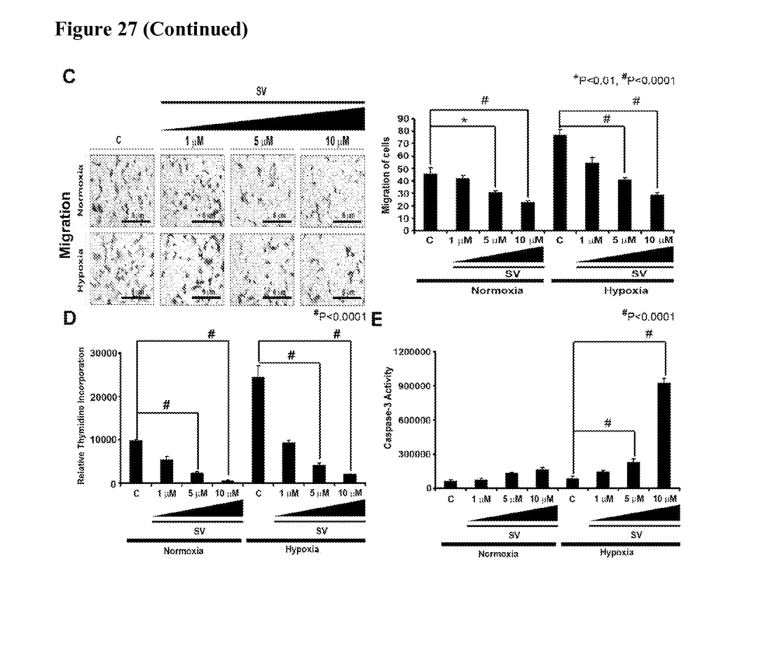

In order to determine whether simvastatin treatment could decrease the conversion of fibroblasts to α-SMA positive cells under hypoxic stress, NIH 3T3 cells were used. The cells were treated with different concentrations of simvastatin (SV) or control (C) and subjected to 24 hours of hypoxia. The expression of α-SMA in the cell lysate was determined using Western blot analysis (FIG. 27A). Semiquantitative analysis was performed, which demonstrated a significant reduction in α-SMA production at 24 hours for 10 μM when compared to controls (P<0.01, Average reduction: 56%).

The synthetic phenotype of the SMC was assessed using confocal imaging for phalloidin and SMA. Confocal microscopy for α-SMA staining was performed on NIH 3T3 cells treated with either simvastatin or control that had been subjected to 24 hours of hypoxia (FIG. 27B). Cells staining red were positive for α-SMA. Cells staining green were positive for phalloidin with the nuclei staining blue. As shown, this demonstrated a significant reduction in α-SMA plus phalloidin staining for the 5 and 10 μM concentrations of simvastatin treated cells when compared to controls for both 24 hours of normoxia (average reduction: 62% (P<0.01), 94% P<0.0001, 5 vs. 10 μM, respectively) and hypoxia (P<0.0001, average reduction: 52%, 82%, 5 vs. 10 μM, respectively).

### Simvastatin Treatment Reduces Migration and Proliferation in Hypoxic Fibroblasts

The following was performed to determine if the migratory capacity of simvastatin treated NIH 3T3 cells was reduced under hypoxia when compared to controls using a matrigel invasion assay. This demonstrated that the migratory capacity of simvastatin treated cells was significantly decreased for all three different concentrations of simvastatin when compared to controls for 24 hours normoxia (5 μM (P<0.01) and 10 μM (P<0.0001) when compared to controls, respectively, average reduction: 32%, 50%) and 24 hours hypoxia (5 μM and 10 μM when compared to controls for 24 hours normoxia, both P<0.0001, respectively, average reduction: 47%, 62%) (FIG. 27C).

The following was performed to determine if the proliferative capacity was decreased as well. Simvastatin treated fibroblasts, when compared to controls that were subjected to hypoxia, exhibited decreased proliferation when compared to controls. NIH 3T3 cells were treated with hypoxia, and a thymidine incorporation assay was performed. This demonstrated that there was significant reduction in the proliferative ability for fibroblasts treated with simvastatin compared to controls for both 24 hours normoxia (5, and 10 μM when compared to controls, all P<0.0001, average reduction: 75%, and 94%) and 24 hours hypoxia (5, and 10 μM when compared to controls, all P<0.0001, average reduction: 83%, and 92%) (FIG. 27D).

Simvastatin Treated Fibroblasts have Increased Caspase 3 Activity

Because there was an increase in TUNEL staining in simvastatin treated vessels when compared to controls, the following was performed to determine if there was an increase in caspase 3 activity. A significant increase in caspase 3 activity was observed when compared to controls (5, and 10 μM when compared to controls, all P<0.0001, average increase: 281%, and 1103%) (FIG. 27E).

Taken together, these results demonstrate that simvastatin treatment results in a significant reduction in VNH by increasing apoptosis, while decreasing cell proliferation and migration mediated through a VEGF-A/MMP-9 pathway. These results also demonstrate that systemic delivery of simvastatin can be used to decrease expression of several important matrix-regulating genes such as VEGF-A, MMP-9, and CTGF. The net result can be an overall decrease in the venous neointimal hyperplasia with a decrease in α-SMA positive cells, migration, proliferation, and an increased apoptosis with positive vascular remodeling. The clinical significance of these results is that it provides rationale for using simvastatin prior to the placement of AVF placement in reducing venous neointimal hyperplasia formation.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing venous stenosis formation of an arteriovenous fistula or graft in a mammal, wherein said method comprises administering a VEGF inhibitor to an adventitia of a vein of said arteriovenous fistula or graft from a position outside said vein under conditions wherein venous stenosis formation of said arteriovenous fistula or graft is reduced.

2. The method of claim 1, wherein said mammal is a human.

3. The method of claim 1, wherein said inhibitor is thalidomide, lapatinib, sunitinib, sorafenib, axitinib, pazopanib, or thiazolidinediones.

4. The method of claim 1, wherein said VEGF inhibitor is administered using a sustained release device positioned outside of said vein.

5. The method of claim 4, wherein said sustained release device comprises chitosan, alginates, polyethylene glycol, poly lactic acid, copoly lactic acid/glycolic acid, dextrans, acrylates, cyclodextrins, caprolactones, block copolymers, or combinations thereof.

6. The method of claim 1, wherein said VEGF inhibitor is administered using a cuff device configured to at least partially surround said vein, wherein said cuff comprises an outlet configured to allow said VEGF inhibitor to be exit said cuff device and contact said adventitia of said vein.

7. The method of claim 6, wherein said cuff device is attached to a pump configured to pump said VEGF inhibitor from a reservoir to said outlet.

8. The method of claim 6, wherein said cuff device comprises polyethylene, polypropylene, polyimides, polyamides, polystyrene, polytetrafluoroethylene (ePTFE), or a combination thereof.

9. The method of claim 1, wherein said VEGF inhibitor is administered using an implantable pump device configured to pump said VEGF inhibitor from a reservoir to an outlet such that said VEGF inhibitor contacts said adventitia of said vein.

10. A method for reducing venous stenosis formation of an arteriovenous fistula or graft in a mammal, wherein said method comprises administering a statin to an adventitia of a vein of said arteriovenous fistula or graft from a position outside said vein under conditions wherein venous stenosis formation of said arteriovenous fistula or graft is reduced.

11. The method of claim 10, wherein said mammal is a human.

12. The method of claim 10, wherein said statin is simvastatin.

13. The method of claim 10, wherein said statin is administered using a sustained release device positioned outside of said vein.

14. The method of claim 13, wherein said sustained release device comprises chitosan, alginates, polyethylene glycol, poly lactic acid, copoly lactic acid/glycolic acid, dextrans, acrylates, cyclodextrins, caprolactones, block copolymers, or combinations thereof.

15. The method of claim 10, wherein said statin is administered using a cuff device configured to at least partially surround said vein, wherein said cuff comprises an outlet configured to allow said statin to be exit said cuff device and contact said adventitia of said vein.

16. The method of claim 15, wherein said cuff device is attached to a pump configured to pump said statin from a reservoir to said outlet.

17. The method of claim 15, wherein said cuff device comprises polyethylene, polypropylene, polyimides, polyamides, polystyrene, polytetrafluoroethylene (ePTFE), or a combination thereof.

18. The method of claim 10, wherein said statin is administered using an implantable pump device configured to pump said statin from a reservoir to an outlet such that said statin contacts said adventitia of said vein.
